(19) United States
(12) Patent Application Publication
(10) Pub. No.: US 2026/0224579 A1
(43) Pub. Date: Aug. 6, 2026

(54) COMPOUNDS AND METHODS FOR INDUCING MHC-I SURFACE EXPRESSION

(71) Applicant: University of Virginia Patent Foundation, Charlottesville, VA (US)

(72) Inventors: Marcos Pires, Crozet, VA (US); Sarah Elizabeth Newkirk, Chesterfield, VA (US); Joseph John Kelly, Gibsonia, PA (US)

(21) Appl. No.: 19/465,052

(22) Filed: Jan. 30, 2026

Related U.S. Application Data
(60) Provisional application No. 63/752,024, filed on Jan. 31, 2025.

(51) Int. Cl. A61K 31/52; A61K 45/06; A61P 35/00; A61P 37/04; C07D 487/04

(52) U.S. Cl. CPC A61K 31/52; A61K 45/06; A61P 35/00; A61P 37/04; C07D 487/04

(57) ABSTRACT

In one aspect, the disclosure relates to compounds capable of affecting major histocompatibility complex class I (MHC-I) expression and methods of using the compounds. This abstract is intended as a scanning tool for purposes of searching in the particular art and is not intended to be limiting of the present disclosure.

### CROSS-REFERENCE TO RELATED APPLICATIONS

This Application claims the benefit of and priority to U.S. Provisional Application No. 63/752,024, filed on Jan. 31, 2025 which is incorporated herein by reference in its entirety.

### BACKGROUND

Immunotherapy has emerged as a powerful strategy for combating cancer by harnessing the patient immune system to recognize and eliminate malignant cells. The major histocompatibility complex class I (MHC-I) has a role in the recognition step. Counteracting cancer immune evasion mechanisms is necessary to enhance the efficacy of immunotherapy treatments. For example, the downregulation of surface MHC-I can directly impair patient response to programmed cell death protein 1 (PD-1) blockade immunotherapy. Despite advances in cancer immunotherapy treatments, there remains a scarcity of compounds that are both potent, efficacious and/or selective inducers of MHC-I surface expression and also effective in the treatment of disorders associated with the loss and/or downregulation of MHC-I. These needs and other needs are satisfied by the present disclosure.

### SUMMARY

In accordance with the purpose(s) of the invention, as embodied and broadly described herein, the invention, in one aspect, relates to compounds such as analogs or derivates of heat shock protein 90 (Hsp90) inhibitors such as purine, resorcinol, and benzoquinone. The disclosed compounds can affect MHC-I expression in a subject or target cell. Also disclosed are methods of making same, pharmaceutical compositions comprising same, kits comprising the same, and methods of treating disorders associated with MHC-I dysfunction and/or uncontrolled cellular proliferation using same.

Disclosed are compounds having a structure represented by a formula (or a pharmaceutically acceptable salt, hydrate, solvate, or polymorph thereof):

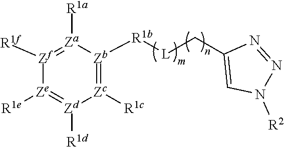

where each of R^{1a}, R^{1b}, R^{1c}, R^{1d}, R^{1e}, and R^{1f} can be characterized by one of the following:
a) each of R^{1a}, R^{1c}, R^{1d}, R^{1e}, and R^{1f} can be independently selected from hydrogen, halogen, hydroxy, an alkyl, and an amine; and R^{1b} is an alkyl or an aryl; or
b) each of R^{1a}, R^{1d}, R^{1e}, and R^{1f} can be independently selected from hydrogen, halogen, hydroxyl, an alkyl, and an amine; and R^{1b} and R^{1c} can be covalently bonded and, together with bridgehead atoms Z^{b} and Z^{c}, comprise an optionally substituted five-membered to eight-membered fused cycloalkyl or aryl;
   where R² can be an alkyl, an alkenyl, an alkynyl, or an aryl; where each of Z^{a}, Z^{b}, Z^{c}, Z^{d}, Z^{e}, and Z^{f} can be independently selected from N and C; where L can be an alkyl, an alkenyl, or an aryl; where m can be 0 or 1; and where n can be an integer ranging from 0 to 10. Also disclosed are pharmaceutical compositions comprising a therapeutically effective amount of one or more disclosed compounds and a pharmaceutically acceptable carrier.

Also disclosed are methods for the treatment of a disorder of uncontrolled cellular proliferation in a mammal comprising the step of administering to the mammal a therapeutically effective amount of at least one disclosed compound or pharmaceutical composition. Also disclosed are methods for the upregulation of MHC-I expression in a mammal comprising the step of administering to the mammal a therapeutically effective amount of at least one disclosed compound or pharmaceutical composition. Also disclosed are methods for the upregulation of MHC-I expression in at least one cell, comprising the step of contacting the cell with an effective amount of at least one disclosed compound or pharmaceutical composition.

Also disclosed are kits comprising at least one disclosed compound or pharmaceutical composition, and one or more of: (a) at least one agent known to increase MHC-I expression; (b) at least one agent known to treat a disorder associated with MHC-I expression; (c) at least one agent known to treat a disease of uncontrolled cellular proliferation; or (d) instructions for treating a disorder associated with MHC-I dysfunction. Also disclosed are uses of a disclosed compound, or a pharmaceutically acceptable salt thereof, or a disclosed pharmaceutical composition.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A shows a schematic representation of a strategy to increase major histocompatibility complex class I (MHC)-I expression and CD8+ T cell response.

FIG. 1B shows a representative workflow diagram illustrating a strategy and process used for the identification and discovery of small molecule MHC-I inducers.

FIG. 2 shows structures of 25 compounds in library of small molecule MHC-I inducers, subdivided by class.

FIG. 3A shows a representative schematic representation of fluorescent antibody readout for increased MHC-I surface expression upon treatment with small molecules inducers.

FIG. 3B shows representative flow cytometry analyses of CT26 cells treated with 1 mM of the indicated compounds (corresponding names and structures found in FIG. 2). The dashed line indicates threshold of 2-fold increase in MHC-I surface expression relative to DMSO control. MFI is the mean fluorescence intensity of the level of fluorescence relative to the DMSO control. Data are represented as mean±SD (n=3). p-values were determined by a two-tailed t-test (* p<0.05, ** p<0.01, *** p<0.001).

FIG. 3C shows representative flow cytometry histograms of CT26 cells incubated with 500 nM of indicated compounds. H-2K^{d} expression was measured by APC anti-mouse H-2K^{d} antibody. The vertical line represents median fluorescence intensity of DMSO treated cells. Data is shown as a representative histogram of the fluorescence intensity of 10,000 events per sample (n=3).

FIG. 4 shows representative flow cytometry analyses of CT26 cells treated with 25-member library at 5 μM. H-2K^{d} surface expression was measured by APC-conjugated anti-mouse H-2K^{d} antibody. MFI means fluorescence intensity of the level of fluorescence relative to the DMSO control. Compounds containing no data bar and denoted with * indicate that the compound was toxic to the cells at 5 μM concentration. Data are represented as mean±SD (n=3).

FIG. 5A shows chemical structures of seven Hsp90 inhibitors tested for their enhancement of MHC-I surface expression.

FIG. 5B shows representative dose-response analyses by flow cytometry of CT26 cells treated with varying concentrations of seven Hsp90 inhibitors. H-2K^{d} expression was measured by APC anti-mouse H-2K^{d} antibody. Data are represented as mean±SD (n=3), and Boltzmann sigmoidal curves were fitted to the data using GraphPad Prism. EC₅₀ values are the concentration of compound needed to achieve 50% of the maximal MHC-I surface expression levels.

FIG. 5C shows representative flow cytometry histograms of HCT116 cells incubated with 200 nM of indicated compounds. HLA-A, B, C surface expression was measured by APC anti-human HLA-A, B, C antibody. The vertical line represents median fluorescence intensity of DMSO treated cells. Data is shown as a representative histogram of the fluorescence intensity of 10,000 events per sample (n=3).

FIG. 5D shows representative flow cytometry analyses of MC38-OVA cells treated with 100 nM of indicated compound. SIINFEKL-H-2K^{b} expression was measured by APC anti-mouse H-2K^{b} bound to SIINFEKL antibody. MFI is the mean fluorescence intensity of the level of fluorescence relative to the DMSO control. Data are represented as mean±SD (n=3). p-values were determined by a two-tailed t-test (* p<0.05, ** p<0.01, **** p<0.0001).

FIG. 6A shows structures 1-8 of azide-containing small molecules from 380 compound library.

FIG. 6B shows chemical structures of triazole products formed from a click reaction between 9-propargyl-2-amino-6-chloropurine and azide compounds 1-8 of 380 library

FIG. 7A shows a representative schematic representation of BIIB021 derivatization strategy.

FIG. 7B shows a representative flow cytometry analysis of CT26 cells treated with click products between 9-propargyl-2-amino-6-chloropurine and the 380-member azide library. H-2K^{d} expression was measured by APC anti-mouse H-2K^{d} antibody and performed in singlet. The dashed lined indicates threshold of 2-fold increase in MHC-I surface expression relative to DMSO control. MFI is the mean fluorescence intensity of the level of fluorescence relative to the DMSO control.

FIG. 7C Stick models of Click MHC-I Booster-325 (CliMB-325, left) and BIIB021 (right) (white) docked into Hsp90 (light orange) were generated using existing crystal structure data (PDB ID: 3qdd) and Rosetta.

FIG. 7D shows a representative dose-response curve and a chemical structure of CliMB-325. CT26 cells were treated with varying concentrations of CliMB-325. H-2K^{d} expression was measured by APC anti-mouse H-2K^{d} antibody via flow cytometry. MFI is the mean fluorescence intensity of the level of fluorescence relative to the DMSO control. Data are represented as mean±SD (n=3), and Boltzmann sigmoidal curves were fitted to the data using GraphPad Prism. EC₅₀ values are the concentration of compound needed to achieve 50% of the maximal MHC-I surface expression levels.

FIG. 7E shows representative dose-response curves of CT26 cells treated with varying concentrations of CliMB-325 or BIIB021 determined via MTT cell viability assay. Data are represented as mean±SD (n=4), and nonlinear regression curves were fitted to the data using GraphPad Prism. CC₅₀ values are the concentration of compound at which maximal cell viability is reduced by 50%.

FIG. 7F shows representative β-galactosidase expression measured in MC38-OVA cells treated with DMSO, BIIB021, or CliMB-325. Data are represented as mean±SD (n=7). p-values were determined by a two-tailed t-test (ns=not significant, **** p<0.0001).

FIG. 7G shows a Venn diagram displaying MHC-I peptides eluted from CT26 cells via mild acid elution that were exclusive to untreated (smaller circle) or CliMB-325-treated (larger circle) cohorts. Peptides which were shared between groups are denoted in the overlapping region. Numbers in italics indicate total peptide numbers, while percentages in bold represent proportion out of 299 total peptides obtained. Data are derived from the same cell culture using 1×10⁷ CT26 cells.

FIG. 8 shows a representative analytical high-performance liquid chromatography (HPLC) of reaction between 9-propargyl-2-amino-6-chloropurine and 2-azido-1-(4-methoxy-phenyl)-ethanone. Overlaid HPLC chromatograms of reaction mixture prior to addition of 2-azido-1-(4-methoxy-phenyl)-ethanone (darker lines) and the full reaction mixture after incubation shaking at 37° C. for 20 h (lighter lines) are shown.

FIG. 9 shows a representative analytical HPLC of reaction between 9-propargyl-2-amino-6-chloropurine and 4-azido-1-butanamine. Overlaid HPLC chromatograms of reaction mixture prior to addition of 4-azido-1-butanamine (darker lines) and the full reaction mixture after incubation shaking at 37° C. for 20 h (lighter lines) are shown.

FIG. 10 shows a representative analytical HPLC of reaction between 9-propargyl-2-amino-6-chloropurine and Boc-4-azido-L-phenylalanine. Overlaid HPLC chromatograms of reaction mixture prior to addition of Boc-4-azido-L-phenylalanine (darker lines) and the full reaction mixture after incubation shaking at 37° C. for 20 h (lighter lines) are shown.

FIG. 11 shows representative flow cytometry analyses of CT26 cells treated with 1 μM 9-propargyl-2-amino-6-chloropurine or a 1:10,000 dilution of CuAAC click reagents (dilution used for FIG. 7B). H-2K^{d} expression was measured by APC anti-mouse H-2K^{d} antibody. MFI is mean fluorescence intensity of the level of fluorescence relative to the DMSO control. Data are represented as mean±SD (n=3). P-values were determined by a two-tailed t-test (ns=not significant).

FIG. 12 shows representative flow cytometry analyses of CT26 cells treated with 1:20,000 dilution of click reaction (500 nM assuming complete conversion) mixtures containing azides 3, 27, 325, and 335. H-2K^{d} expression was measured by APC anti-mouse H-2K^{d} antibody. MFI means fluorescence intensity of the level of fluorescence relative to the DMSO control. Data are represented as mean±SD (n=3). p-values were determined by a two-tailed t-test (*** p<0.001).

FIG. 13 shows a representative dose-response curve and chemical structure of the regioisomer of CliMB-325. CT26 cells were treated with varying concentrations of the CliMB-325 regioisomer. H-2K^{d} expression was measured by APC anti-mouse H-2K^{d} antibody via flow cytometry. MFI means fluorescence intensity of the level of fluorescence relative to the DMSO control. Data are represented as mean±SD (n=3), and Boltzmann sigmoidal curves were fitted to the data using GraphPad Prism. EC₅₀ values are the concentration of compound needed to achieve 50% of the maximal MHC-I surface expression levels.

FIG. 14 shows representative length distributions of MHC-I peptides isolated by mild acid elution (MAE) from CT26 cells treated with (right of horizontal tick-mark) or without (left of horizontal tick mark) CliMB-325. Data in displayed histograms are derived from a single cell culture, and peptides were obtained from 1×10⁷ cells per sample. The shaded area indicates the in silico length filter of 8 to 14 amino acids that was applied for all analyses (except for this figure). The summed fraction of 8- to 14-mers among all peptides was 64% and 66% for untreated and CliMB-325-treated samples, respectively

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Aspects of the present disclosure will employ, unless otherwise indicated, techniques of molecular biology, microbiology, organic chemistry, biochemistry, physiology, cell biology, blood vessel biology, and the like, which are within the skill of the art. Such techniques are explained fully in the literature.

Prior to describing the various aspects of the present disclosure, the following definitions are provided and should be used unless otherwise indicated. Additional terms may be defined elsewhere in the present disclosure.

### A. DEFINITIONS

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used herein, the term “and/or” includes any and all combinations of one or more of the associated listed items. Expressions such as “at least one of,” when preceding a list of elements, modify the entire list of elements and do not modify the individual elements of the list.

As used in the specification and the appended claims, the singular forms “a,” “an” and “the” include plural referents unless the context clearly dictates otherwise. Thus, for example, reference to “an agent”, “a carrier,” or “a disorder,” including, but not limited to, two or more such agents, carriers, or disorders, including combinations of agents, carriers, or disorders, and the like.

Reference to “a/an” chemical compound, protein, and antibody each refers to one or more molecules of the chemical compound, protein, and antibody rather than being limited to a single molecule of the chemical compound, protein, and antibody. Furthermore, the one or more molecules may or may not be identical, so long as they fall under the category of the chemical compound, protein, and antibody. Thus, for example, “an” antibody is interpreted to include one or more antibody molecules of the antibody, where the antibody molecules may or may not be identical (e.g., different isotypes and/or different antigen binding sites as may be found in a polyclonal antibody).

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, “about,” “approximately,” “substantially,” and the like, when used in connection with a numerical variable, can generally refers to the value of the variable and to all values of the variable that are within the experimental error (e.g., within the 95% confidence interval for the mean) or within +/−10% of the indicated value, whichever is greater. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro- drugs, which become biologically active or more active after they have been placed in a predetermined physiological environment.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and juvenile subjects, whether male or female, are intended to be covered. In one aspect, the subject is a mammal. A patient refers to a subject afflicted with a disease or disorder. The term “patient” includes human and veterinary subjects.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as cancer, a disorder or disease associate with uncontrolled cellular proliferation, and/or a disorder or disease associated with the loss and/or downregulation of MHC-I. The effect can be therapeutic in terms of a partial or complete cure of a disease, condition, symptom or adverse effect attributed to the disease, disorder, or condition. The term “treatment” as used herein can include any treatment of cancer, a disorder or disease associate with uncontrolled cellular proliferation, and/or a disorder or disease associated with the loss and/or downregulation of MHC-I in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound and/or pharmaceutical composition, for example, can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

The term “pharmaceutically acceptable” describes a material that is not biologically or otherwise undesirable, i.e., without causing an unacceptable level of undesirable biological effects or interacting in a deleterious manner.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

The term “pharmaceutically acceptable ester” refers to esters of compounds of the present disclosure which hydrolyze in vivo and include those that break down readily in the human body to leave the parent compound or a salt thereof. Examples of pharmaceutically acceptable, non-toxic esters of the present disclosure include C 1 -to- C 6 alkyl esters and C 5 -to- C 7 cycloalkyl esters, although C 1 -to- C 4 alkyl esters are preferred. Esters of disclosed compounds can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, for example with methyl iodide, benzyl iodide, cyclopentyl iodide or alkyl triflate. They also can be prepared by reaction of the compound with an acid such as hydrochloric acid and an alcohol such as ethanol or methanol.

The term “pharmaceutically acceptable amide” refers to non-toxic amides of the present disclosure derived from ammonia, primary C 1 -to- C 6 alkyl amines and secondary C 1 -to- C 6 dialkyl amines. In the case of secondary amines, the amine can also be in the form of a 5- or 6-membered heterocycle containing one nitrogen atom. Amides derived from ammonia, C 1 -to- C3 alkyl primary amides and C 1 -to- C 2 dialkyl secondary amides are preferred. Amides of disclosed compounds can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable amides are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, and piperidine. They also can be prepared by reaction of the compound with an acid such as sulfuric acid and an alkylcarboxylic acid such as acetic acid, or with acid and an arylcarboxylic acid such as benzoic acid under dehydrating conditions such as with molecular sieves added. The composition can contain a compound of the present disclosure in the form of a pharmaceutically acceptable prodrug.

The term “pharmaceutically acceptable prodrug” or “prodrug” represents those prodrugs of the compounds of the present disclosure which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the present disclosure can be rapidly transformed in vivo to a parent compound having a structure of a disclosed compound, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987).

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

The term “contacting” as used herein refers to bringing a disclosed compound or pharmaceutical composition in proximity to a cell, a target protein, or other biological entity together in such a manner that the disclosed compound or pharmaceutical composition can affect the activity of the a cell, target protein, or other biological entity, either directly; i.e., by interacting with the cell, target protein, or other biological entity itself, or indirectly; i.e., by interacting with another molecule, co-factor, factor, or protein on which the activity of the cell, target protein, or other biological entity itself is dependent. In some instances, contacting can comprise both physical and chemical interactions between the indicated components. It is to be understood that chemical interactions can comprise a combination of covalent and non-covalent interactions, including one or more of ionic, dipolar, van der Waals interactions, and the like.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

It is understood, that unless otherwise specified, temperatures referred to herein are based on atmospheric pressure (i.e. one atmosphere).

Described herein are compounds that have therapeutic or clinical utility. Also described herein are methods of synthesizing the compounds. Also described herein are methods of administering the compounds to a subject in need thereof. In some aspects, the subject can have a need for treatment of a disease or disorder associated with uncontrolled cellular proliferation or the loss and/or downregulation of MHC-I. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, or thiol as described herein.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “alkenyl” as used herein is a hydrocarbon group of from 2 to 24 carbon atoms with a structural formula containing at least one carbon-carbon double bond. The alkenyl group can be cyclic or acyclic. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “heterocycle” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl to two aryl groups that are bound together via a fused ring structure, as in naphthalene, or are attached via one or more carbon-carbon bonds, as in biphenyl.

The term “heteroaryl” as used herein refers to an aromatic group that has at least one heteroatom incorporated within the ring of the aromatic group. The term “heteroaryl” is a type of aryl group as defined above, and is included within the meaning of the term “aryl,” Examples of heteroatoms include, but are not limited to, nitrogen, oxygen, sulfur, and phosphorus, where N- oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above.

The term “hydroxyl” or “hydroxy” as used herein is represented by the formula —OH.

The term “nitrile” or “cyano” as used herein is represented by the formula —CN.

The terms “amine” or “amino” as used herein are represented by the formula —NA¹A², where A¹and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

### B. ABBREVIATIONS

### MHC-I Major Histocompatibility Class I

### C. COMPOUNDS

Counteracting cancer immune evasion mechanisms is important in the effort to enhance the efficacy of immunotherapy treatments. For example, a significant portion of patients fail to respond to PD-1 blockade therapy due to the development of resistant tumors resulting from MHC-I downregulation. This challenge underscores the need to reengage the immune system by converting immunologically ‘cold’ tumors back into ‘hot’ tumors that can be recognized and targeted for elimination. Therefore, one avenue for combating resistant cancer is increasing MHC-I surface expression. In one aspect, disclosed herein are compounds that can affect MHC-I expression, such as by upregulation. In another aspect, the disclosed compounds can be used in the treatment of cancer, a disorder or disease associated with uncontrolled cellular proliferation, and/or a disorder or disease associated with MHC-I dysfunction.

In a further aspect, the disclosed compounds have a structure represented by a formula (or a pharmaceutically acceptable salt, hydrate, solvate, or polymorph thereof):

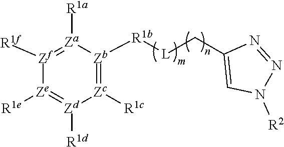

where each of R^{1a}, R^{1b}, R^{1c}, R^{1d}, R^{1e}, and R^{1f} can be characterized by one of the following:
c) each of R^{1a}, R^{1c}, R^{1d}, R^{1e}, and R^{1f} can be independently selected from hydrogen, halogen, hydroxy, an alkyl, and an amine; and R^{1b} is an alkyl or an aryl; or
d) each of R^{1a}, R^{1d}, R^{1e}, and R^{1f} can be independently selected from hydrogen, halogen, hydroxyl, an alkyl, and an amine; and R^{1b} and R^{1c} can be covalently bonded and, together with bridgehead atoms Z^{b} and Z^{c}, comprise an optionally substituted five-membered to eight-membered fused cycloalkyl or aryl;
   where R² can be an alkyl, an alkenyl, an alkynyl, or an aryl; where each of Z^{a}, Z^{b}, Z^{c}, Z^{d}, Z^{e}, and Z^{f} can be independently selected from N and C; where L can be an alkyl, an alkenyl, or an aryl; where m can be 0 or 1. In one aspect, n can be an integer ranging from 0 to 10, from 0 to 8, from 0 to 6, from 0 to 4, from 1 to 10, from 1 to 8, from 1 to 6, or from 1 to 4. In another aspect, n can be 0.

In another aspect, each of R^{1a}, R^{1c}, R^{1d}, R^{1e}, and R^{1f} can be independently selected from hydrogen, halogen, a C1-C6 alkyl, and —NR¹⁰R¹¹, wherein each of R₁₀ and R¹¹ is independently selected from hydrogen and a C1-C6 alkyl. In another aspect, each of R^{1a}, R^{1c}, R^{1d}, R^{1e}, and R^{1f} can be independently selected from hydrogen, hydroxy, and a C1-C6 alkyl. In another aspect, each of R^{1a}, R^{1d}, R^{1e}, and R^{1f} can be independently selected from hydrogen, halogen, a C1-C6 alkyl, and —NR¹⁰R¹¹, wherein each of R¹⁰ and R¹¹ is independently selected from hydrogen and a C1-C6 alkyl. In another aspect, each of R^{1a}, R^{1d}, R^{1e}, and R^{1f} can be independently selected from hydrogen, hydroxy, and a C1-C6 alkyl. In another aspect, R^{1b} can be a C1-C10 alkyl or a C5-C8 aryl. In another aspect, R^{1b} and R^{1c} can be covalently bonded and, together with bridgehead atoms Z^{b} and Z^{c}, comprise a five-membered to eight-membered fused aryl comprising at least one heteroatom. In another aspect, at least two of Z^{a}, Z^{b}, Z^{c}, Z^{d}, Z^{e}, and Z^{f} can be N.

In another aspect, R² can be a C1-C10 acyclic alkyl, a C3-C12 cycloalkyl, a C1-C10 acyclic alkenyl, C3-C12 cycloalkenyl, or a C5-C8 aryl. In a further aspect, R² can be further substituted with halogen, hydroxy, alkoxy, nitrile, an amine, an alkyl, an alkenyl, or an aryl. In another aspect, L can be a C1-C6 acyclic alkyl, a C1-C6 alkenyl, or a C5-C8 aryl.

In another aspect, the compound can have a structure represented by a formula:

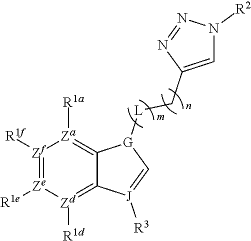

where R^{1a}, R^{1d}, R^{1e}, R^{1f}, R², Z^{a}, Z^{d}, Z^{e}, Z^{f}, L, m, and n can be as defined previously. In another aspect, R³ can be hydrogen, halogen, an alkyl, or an aryl; G can be C or N; and J can be C or N. In a further aspect, R³ can be hydrogen, halogen, a C1-C10 acyclic alkyl, or a C5-C8 aryl. In another aspect, R³ can be a C1-C10 acyclic alkyl or a C5-C8 aryl. R³ can be further substituted with halogen, hydroxy, alkoxy, nitrile, an amine, an alkyl, an alkenyl, or an aryl.

In various aspects, it is contemplated herein that the disclosed compounds further comprise their bioisosteric equivalents. The term “bioisosteric equivalent” refers to compounds or groups that possess near equal molecular shapes and volumes, approximately the same distribution of electrons, and which exhibit similar physical and biological properties. Examples of such equivalents are: (i) fluorine vs. hydrogen, (ii) oxo vs. thia, (iii) hydroxyl vs. amide, (iv) carbonyl vs. oxime, (v) carboxylate vs. tetrazole. Examples of such bioisosteric replacements can be found in the literature and examples of such are: (i) Burger A, Relation of chemical structure and biological activity; in Medicinal Chemistry Third ed., Burger A, ed.; Wiley-Interscience; New York, 1970, 64-80; (ii) Burger, A.; “Isosterism and bioisosterism in drug design”; Prog. Drug Res. 1991, 37, 287-371; (iii) Burger A, “Isosterism and bioanalogy in drug design”, Med. Chem. Res. 1994, 4, 89-92; (iv) Clark R D, Ferguson A M, Cramer R D, “Bioisosterism and molecular diversity”, Perspect. Drug Discovery Des. 1998, Sep. 10, 2011, 213-224; (v) Koyanagi T, Haga T, “Bioisosterism in agrochemicals”, ACS Symp. Ser. 1995, 584, 15-24; (vi) Kubinyi H, “Molecular similarities. Part 1. Chemical structure and biological activity”, Pharm. Unserer Zeit 1998, 27, 92-106; (vii) Lipinski C A.; “Bioisosterism in drug design”; Annu. Rep. Med. Chem. 1986, 21, 283-91; (viii) Patani G A, LaVoie E J, “Bioisosterism: A rational approach in drug design”, Chem. Rev. (Washington, D.C.) 1996, 96, 3147-3176; (ix) Soskic V, Joksimovic J, “Bioisosteric approach in the design of new dopaminergic/serotonergic ligands”, Curr. Med. Chem. 1998, 5, 493-512 (x) Thornber C W, “Isosterism and molecular modification in drug design”, Chem. Soc. Rev. 1979, 8, 563-80.

In further aspects, bioisosteres are atoms, ions, or molecules in which the peripheral layers of electrons can be considered substantially identical. The term bioisostere is usually used to mean a portion of an overall molecule, as opposed to the entire molecule itself. Bioisosteric replacement involves using one bioisostere to replace another with the expectation of maintaining or slightly modifying the biological activity of the first bioisostere. The bioisosteres in this case are thus atoms or groups of atoms having similar size, shape and electron density. Preferred bioisosteres of esters, amides or carboxylic acids are compounds containing two sites for hydrogen bond acceptance. In one embodiment, the ester, amide or carboxylic acid bioisostere is a 5-membered monocyclic heteroaryl ring, such as an optionally substituted 1H-imidazolyl, an optionally substituted oxazolyl, 1H-tetrazolyl, [1,2,4]triazolyl, or an optionally substituted [1,2,4]oxadiazolyl.

In various aspects, it is contemplated herein that the disclosed compounds further comprise their isotopically-labelled or isotopically-substituted variants, i.e., compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

In various aspects, the disclosed compounds can possess at least one center of asymmetry, they can be present in the form of their racemates, in the form of the pure enantiomers and/or diastereomers or in the form of mixtures of these enantiomers and/or diastereomers. The stereoisomers can be present in the mixtures in any arbitrary proportions. In some aspects, provided this is possible, the disclosed compounds can be present in the form of the tautomers.

Thus, methods which are known per se can be used, for example, to separate the disclosed compounds which possess one or more chiral centers and occur as racemates into their optical isomers, i.e., enantiomers or diastereomers. The separation can be effected by means of column separation on chiral phases or by means of recrystallization from an optically active solvent or using an optically active acid or base or by means of derivatizing with an optically active reagent, such as an optically active alcohol, and subsequently cleaving off the residue.

In various aspects, the disclosed compounds can be in the form of a co-crystal. The term “co-crystal” means a physical association of two or more molecules which owe their stability through non-covalent interaction. One or more components of this molecular complex provide a stable framework in the crystalline lattice. In certain instances, the guest molecules are incorporated in the crystalline lattice as anhydrates or solvates, see e.g. “Crystal Engineering of the Composition of Pharmaceutical Phases. Do Pharmaceutical Co-crystals Represent a New Path to Improved Medicines?” Almarasson, O., et. al., The Royal Society of Chemistry, 1889-1896, 2004. Preferred co-crystals include p-toluenesulfonic acid and benzenesulfonic acid.

The term “pharmaceutically acceptable co-crystal” means one that is compatible with the other ingredients of the formulation and not deleterious to the recipient thereof.

In a further aspect, the disclosed compounds can be isolated as solvates and, in particular, as hydrates of a disclosed compound, which can be obtained, for example, by crystallization from a solvent or from aqueous solution. In this connection, one, two, three or any arbitrary number of solvate or water molecules can combine with the compounds according to the invention to form solvates and hydrates.

The disclosed compounds can be used in the form of salts derived from inorganic or organic acids. Pharmaceutically acceptable salts include salts of acidic or basic groups present in the disclosed compounds. Suitable pharmaceutically acceptable salts include base addition salts, including alkali metal salts, e.g., sodium or potassium salts; alkaline earth metal salts, e.g., calcium or magnesium salts; and salts formed with suitable organic ligands, e.g., quaternary ammonium salts, which may be similarly prepared by reacting the drug compound with a suitable pharmaceutically acceptable base. The salts can be prepared in situ during the final isolation and purification of the compounds of the present disclosure; or following final isolation by reacting a free base function, such as a secondary or tertiary amine, of a disclosed compound with a suitable inorganic or organic acid; or reacting a free acid function, such as a carboxylic acid, of a disclosed compound with a suitable inorganic or organic base.

Acidic addition salts can be prepared in situ during the final isolation and purification of a disclosed compound, or separately by reacting moieties comprising one or more nitrogen groups with a suitable acid. In various aspects, acids which may be employed to form pharmaceutically acceptable acid addition salts include such inorganic acids as hydrochloric acid, sulphuric acid and phosphoric acid and such organic acids as oxalic acid, maleic acid, succinic acid and citric acid. In a further aspect, salts further include, but are not limited, to the following: hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate, butyrate, camphorate, camphorsulfonate, digluconate, glycerophosphate, hemisulfate, heptanoate, hexanoate, fumarate, hydrochloride, 2-hydroxyethanesulfonate (isethionate), nicotinate, 2-naphthalenesulfonate, oxalate, pectinate, persulfate, 3-phenylpropionate, picrate, pivalate, propionate, succinate, tartrate, thiocyanate, phosphate, glutamate, bicarbonate, undecanoate, and pamoate (i.e., 1,1′-methylene-bis-(2-hydroxy-3-naphthoate)) salts. Also, basic nitrogen-containing groups can be quatemized with such agents as lower alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others.

Basic addition salts can be prepared in situ during the final isolation and purification of a disclosed compound, or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like. In further aspects, bases which may be used in the preparation of pharmaceutically acceptable salts include the following: ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

### D. PHARMACEUTICAL COMPOSITIONS

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitonealy, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In various aspects, a disclosed compound comprising an acidic group or moiety, e.g., a carboxylic acid group, can be used to prepare a pharmaceutically acceptable salt. For example, such a disclosed compound may comprise an isolation step comprising treatment with a suitable inorganic or organic base. In some cases, it may be desirable in practice to initially isolate a compound from the reaction mixture as a pharmaceutically unacceptable salt and then simply convert the latter back to the free acid compound by treatment with an acidic reagent, and subsequently convert the free acid to a pharmaceutically acceptable base addition salt. These base addition salts can be readily prepared using conventional techniques, e.g., by treating the corresponding acidic compounds with an aqueous solution containing the desired pharmacologically acceptable cations and then evaporating the resulting solution to dryness, preferably under reduced pressure. Alternatively, they also can be prepared by mixing lower alkanolic solutions of the acidic compounds and the desired alkali metal alkoxide together, and then evaporating the resulting solution to dryness in the same manner as before.

Bases which can be used to prepare the pharmaceutically acceptable base-addition salts of the base compounds are those which can form non-toxic base-addition salts, i.e., salts containing pharmacologically acceptable cations such as, alkali metal cations (e.g., lithium, potassium and sodium), alkaline earth metal cations (e.g., calcium and magnesium), ammonium or other water-soluble amine addition salts such as N-methylglucamine-(meglumine), lower alkanolammonium and other such bases of organic amines. In a further aspect, derived from pharmaceutically acceptable organic non-toxic bases include primary, secondary, and tertiary amines, as well as cyclic amines and substituted amines such as naturally occurring and synthesized substituted amines. In various aspects, such pharmaceutically acceptable organic non-toxic bases include, but are not limited to, ammonia, methylamine, ethylamine, propylamine, isopropylamine, any of the four butylamine isomers, betaine, caffeine, choline, dimethylamine, diethylamine, diethanolamine, dipropylamine, diisopropylamine, di-n-butylamine, N,N′-dibenzylethylenediamine, pyrrolidine, piperidine, morpholine, trimethylamine, triethylamine, tripropylamine, tromethamine, 2-diethylaminoethanol, 2-dimethylaminoethanol, ethanolamine, quinuclidine, pyridine, quinoline and isoquinoline; benzathine, N-methyl-D-glucamine, ethylenediamine, N-ethylmorpholine, N-ethylpiperidine, glucamine, glucosamine, methylglucamine, morpholine, piperazine, piperidine, polyamine resins, procaine, purines, theobromine, hydrabamine salts, and salts with amino acids such as, for example, histidine, arginine, lysine and the like. The foregoing salt forms can be converted by treatment with acid back into the free acid form.

In various aspects, a disclosed compound comprising a protonatable group or moiety, e.g., an amino group, can be used to prepare a pharmaceutically acceptable salt. For example, such a disclosed compound may comprise an isolation step comprising treatment with a suitable inorganic or organic acid. In some cases, it may be desirable in practice to initially isolate a compound from the reaction mixture as a pharmaceutically unacceptable salt and then simply convert the latter back to the free base compound by treatment with a basic reagent, and subsequently convert the free base to a pharmaceutically acceptable acid addition salt. These acid addition salts can be readily prepared using conventional techniques, e.g., by treating the corresponding basic compounds with an aqueous solution containing the desired pharmacologically acceptable anions and then evaporating the resulting solution to dryness, preferably under reduced pressure. Alternatively, they also can be prepared by treating the free base form of the disclosed compound with a suitable pharmaceutically acceptable non-toxic inorganic or organic acid.

Acids which can be used to prepare the pharmaceutically acceptable acid-addition salts of the base compounds are those which can form non-toxic acid-addition salts, i.e., salts containing pharmacologically acceptable anions formed from their corresponding inorganic and organic acids. Exemplary, but non-limiting, inorganic acids include hydrochloric hydrobromic, sulfuric, nitric, phosphoric and the like. Exemplary, but non-limiting, organic acids include acetic, benzenesulfonic, benzoic, camphorsulfonic, citric, ethanesulfonic, fumaric, gluconic, glutamic, isethionic, lactic, maleic, malic, mandelicmethanesulfonic, mucic, pamoic, pantothenic, succinic, tartaric, p-toluenesulfonic acid and the like. In a further aspect, the acid-addition salt comprises an anion formed from hydrobromic, hydrochloric, maleic, phosphoric, sulfuric, and tartaric acids.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques.

The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate,-phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate,-triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require upregulation of MHC-I expression, an appropriate dosage level will generally be about 0.01 to 1000 mg per kg patient body weight per day and can be administered in single or multiple doses. In various aspects, the dosage level will be about 0.1 to about 500 mg/kg per day, about 0.1 to 250 mg/kg per day, or about 0.5 to 100 mg/kg per day. A suitable dosage level can be about 0.01 to 1000 mg/kg per day, about 0.01 to 500 mg/kg per day, about 0.01 to 250 mg/kg per day, about 0.05 to 100 mg/kg per day, or about 0.1 to 50 mg/kg per day. Within this range the dosage can be 0.05 to 0.5, 0.5 to 5.0 or 5.0 to 50 mg/kg per day. For oral administration, the compositions are preferably provided in the form of tablets containing 1.0 to 1000 mg of the active ingredient, particularly 1.0, 5.0, 10, 15, 20, 25, 50, 75, 100, 150, 200, 250, 300, 400, 500, 600, 750, 800, 900 and 1000 mg of the active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The present disclosure is further directed to a method for the manufacture of a medicament for modulating MHC-I expression (e.g., treatment of one or more diseases or disorders associated with MHC-I dysfunction) in mammals (e.g., humans) comprising combining one or more disclosed compounds, products, or compositions with a pharmaceutically acceptable carrier or diluent. Thus, in one aspect, the present disclosure further relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological or clinical conditions.

It is understood that the disclosed compositions can be prepared from the disclosed compounds. It is also understood that the disclosed compositions can be employed in the disclosed methods of using.

As already mentioned, the present disclosure relates to a pharmaceutical composition comprising a therapeutically effective amount of a disclosed compound, a product of a disclosed method of making, a pharmaceutically acceptable salt, a hydrate thereof, a solvate thereof, a polymorph thereof, and a pharmaceutically acceptable carrier. Additionally, the present disclosure relates to a process for preparing such a pharmaceutical composition, characterized in that a pharmaceutically acceptable carrier is intimately mixed with a therapeutically effective amount of a compound according to the present disclosure.

As already mentioned, the present disclosure also relates to a pharmaceutical composition comprising a disclosed compound, a product of a disclosed method of making, a pharmaceutically acceptable salt, a hydrate thereof, a solvate thereof, a polymorph thereof, and one or more other drugs in the treatment, prevention, control, amelioration, or reduction of risk of diseases or conditions for a disclosed compound or the other drugs may have utility as well as to the use of such a composition for the manufacture of a medicament. The present disclosure also relates to a combination of disclosed compound, a product of a disclosed method of making, a pharmaceutically acceptable salt, a hydrate thereof, a solvate thereof, or a polymorph thereof. The present disclosure also relates to such a combination for use as a medicine. The present disclosure also relates to a product comprising (a) disclosed compound, a product of a disclosed method of making, a pharmaceutically acceptable salt, a hydrate thereof, a solvate thereof, a polymorph thereof, and (b) an additional therapeutic agent, as a combined preparation for simultaneous, separate or sequential use in the treatment or prevention of a condition in a mammal, including a human, the treatment or prevention of which is affected or facilitated by the modulatory effect of the disclosed compound and the additional therapeutic agent. The different drugs of such a combination or product may be combined in a single preparation together with pharmaceutically acceptable carriers or diluents, or they may each be present in a separate preparation together with pharmaceutically acceptable carriers or diluents.

### E. METHODS OF USING THE COMPOUNDS

In a further aspect, the present disclosure provides methods of treatment comprising administration of a therapeutically effective amount of a disclosed compound or pharmaceutical composition as disclosed herein above to a subject in need thereof. The disclosed compounds can be used as single agents or in combination with one or more other drugs in the treatment, prevention, control, amelioration, or reduction of risk of the aforementioned diseases, disorders and conditions for which the compounds disclosed herein or the other drugs have utility, where the combination of drugs together are safer or more effective than either drug alone. The other drug(s) can be administered by a route and in an amount commonly used therefore, contemporaneously, or sequentially with a disclosed compound. When a disclosed compound is used contemporaneously with one or more other drugs, a pharmaceutical composition in unit dosage form containing such drugs and the disclosed compound is preferred. However, the combination therapy can also be administered on overlapping schedules. It is also envisioned that the combination of one or more active ingredients and a disclosed compound will be more efficacious than either as a single agent.

In one aspect, the compounds and pharmaceutical compositions disclosed herein are useful for treating, preventing, ameliorating, controlling, or reducing the risk of disorders of uncontrolled cellular proliferation in a subject. The method of treatment of such disorders can include the step of administering to a subject a therapeutically effective amount of at least one compound disclosed herein or administering to a subject a pharmaceutical composition as disclosed herein. The subject can be a mammal, such as a human. In one aspect, the method of treatment can further comprise identifying a subject in need of treatment of a disorder of uncontrolled cellular proliferation. In another aspect, the subject can be diagnosed with a need for treatment prior to the administering step.

In a further aspect, the disorder of uncontrolled cellular proliferation can be associated with MHC-I dysfunction (e.g., MHC-I downregulation). In another aspect, the disorder can be a cancer. Examples of cancer include, but are not limited to, colorectal cancer, esophageal cancer, bladder cancer, lung cancer, laryngeal cancer, breast cancer, skin cancer, and throat cancer. In a further aspect, the disorder is a colorectal cancer, bladder cancer, lung cancer, laryngeal cancer, breast cancer, melanoma, or head and neck squamous cell carcinoma.

Also disclosed herein is a method for upregulation of MHC-I expression in a subject. The method can include the step of administering to the subject a therapeutically effective amount of at least one compound disclosed herein or administering to the subject a pharmaceutical composition disclosed herein. In one aspect, the subject is a mammal, such as a human. In one aspect, the method of inhibition can further comprise identifying a subject in need upregulation of MHC-I expression and/or activity. In another aspect, the subject can be diagnosed with a need for upregulation of MHC-I expression prior to the administering step.

Also disclosed herein is a method for upregulation of MHC-I expression in at least one cell. The method can include the step of contacting the at least one cell with an effective amount of at least one compound disclosed herein or at least one pharmaceutical composition disclosed herein. The cell can be mammalian, such as human. In one aspect, the cell can be isolated from a subject prior to the contacting step. In another aspect, the contacting can be done via administration to a subject. In one aspect, the method of inhibiting and/or modulating can further comprise identifying a subject in need for upregulation of MHC-I expression. In another aspect, the subject can be diagnosed with a need for upregulation of MHC-I expression prior to the contacting step.

In a further aspect, contacting the at least one cell treats a disorder. The disorder can be a cancer. Examples of cancer include, but are not limited to, colorectal cancer, esophageal cancer, bladder cancer, lung cancer, laryngeal cancer, breast cancer, skin cancer, and throat cancer. In a further aspect, the disorder is a colorectal cancer, bladder cancer, lung cancer, laryngeal cancer, breast cancer, melanoma, or head and neck squamous cell carcinoma.

### F. KITS

In a further aspect, the present disclosure relates to kits comprising at least one disclosed compound, or a pharmaceutically acceptable salt, hydrate, solvate, or polymorph thereof, and one or more of: (a) at least one agent known to increase MHC-I expression; (b) at least one agent known to treat a disorder associated with MHC-I expression; (c) at least one agent known to treat a disease of uncontrolled cellular proliferation; or (d) instructions for treating a disorder associated with MHC-I dysfunction. In one aspect, the disorder associated with MHC-I expression or dysfunction can be a cancer. Examples of cancer include, but are not limited to, colorectal cancer, esophageal cancer, bladder cancer, lung cancer, laryngeal cancer, breast cancer, skin cancer, and throat cancer. In a further aspect, the disorder is a colorectal cancer, bladder cancer, lung cancer, laryngeal cancer, breast cancer, melanoma, or head and neck squamous cell carcinoma.

The disclosed compounds and/or pharmaceutical compositions comprising the disclosed compounds can conveniently be presented as a kit, whereby two or more components, which may be active or inactive ingredients, carriers, diluents, and the like, are provided with instructions for preparation of the actual dosage form by the patient or person administering the drug to the patient. Such kits may be provided with all necessary materials and ingredients contained therein, or they may contain instructions for using or making materials or components that must be obtained independently by the patient or person administering the drug to the patient. In further aspects, a kit can include optional components that aid in the administration of the unit dose to patients, such as vials for reconstituting powder forms, syringes for injection, customized IV delivery systems, inhalers, etc. Additionally, a kit can contain instructions for preparation and administration of the compositions. The kit can be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

In a further aspect, the disclosed kits can be packaged in a daily dosing regimen (e.g., packaged on cards, packaged with dosing cards, packaged on blisters or blow-molded plastics, etc.). Such packaging promotes products and increases patient compliance with drug regimens. Such packaging can also reduce patient confusion. The present invention also features such kits further containing instructions for use.

In a further aspect, the present disclosure also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration.

In various aspects, the disclosed kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using or treating, and/or the disclosed compositions.

### G. ASPECTS

The following listing of exemplary aspects supports and is supported by the disclosure provided herein.

Aspect 1. A compound having a structure represented by a formula:

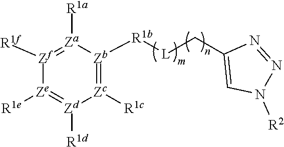

wherein each of R^{1a}, R^{1b}, R^{1c}, R^{1d}, R^{1e}, and R^{1f} are characterized by one of the following:
a) each of R^{1a}, R^{1c}, R^{1d}, R^{1e}, and R^{1f} is independently selected from hydrogen, halogen, hydroxy, an alkyl, and an amine; and R^{1b} is an alkyl or an aryl; or b) each of R^{1a}, R^{1d}, R^{1e}, and R^{1f} is independently selected from hydrogen, halogen, hydroxyl, an alkyl, and an amine; and R^{1b} and R^{1c} are covalently bonded and, together with bridgehead atoms Z^{b} and Z^{c}, comprise a five-membered to eight-membered fused cycloalkyl or aryl; wherein R² is an alkyl, an alkenyl, an alkynyl, or an aryl; wherein each of Z^{a}, Z^{b}, Z^{c}, Z^{d}, Z^{e}, and Z^{f} is independently selected from N and C; wherein L is an alkyl, an alkenyl, or an aryl; wherein m is 0 or 1; and wherein n is an integer ranging from 0 to 10; or a pharmaceutically acceptable salt, solvate, or polymorph thereof.

Aspect 2. The compound of aspect 1, wherein each of R^{1a}, R^{1c}, R^{1d}, R^{1e}, and R^{1f} is independently selected from hydrogen, halogen, a C1-C6 alkyl, and —NR¹⁰R¹¹, wherein each of R¹⁰ and R¹¹ is independently selected from hydrogen and a C1-C6 alkyl, and wherein and R^{1b} is an alkyl or an aryl.

Aspect 3. The compound of aspect 1, wherein each of R^{1a}, R^{1c}, R^{1d}, R^{1e}, and R^{1f} is independently selected from hydrogen, hydroxy, and a C1-C6 alkyl and wherein and R^{1b} is an alkyl or an aryl.

Aspect 4. The compound of aspect 1, wherein each of R^{1a}, R^{1d}, R^{1e}, and R^{1f} is independently selected from hydrogen, halogen, a C1-C6 alkyl, and —NR¹⁰R¹¹, wherein each of R¹⁰ and R¹¹ is independently selected from hydrogen and a C1-C6 alkyl and wherein R^{1b} and R^{1c} are covalently bonded and, together with bridgehead atoms Z^{b} and Z^{c}, comprise a five-membered to eight-membered fused cycloalkyl or aryl.

Aspect 5. The compound of aspect 1, wherein each of R^{1a}, R^{1d}, R^{1e}, and R^{1f} is independently selected from hydrogen, hydroxy, and a C1-C6 alkyl and wherein R^{1b} and R^{1c} are covalently bonded and, together with bridgehead atoms Z^{b} and Z^{c}, comprise a five-membered to eight-membered fused cycloalkyl or aryl.

Aspect 6. The compound of any one of aspects 1-3, wherein R^{1b} is a C1-C10 alkyl or a C5-C8 aryl.

Aspect 7. The compound of any one of aspects 1, 4, or 5, wherein R^{1b} and R^{1c} are covalently bonded and, together with bridgehead atoms Z^{b} and Z^{c}, comprise a five-membered to eight-membered fused aryl comprising at least one heteroatom.

Aspect 8. The compound of any one of aspects 1-7, wherein R² is a C1-C10 acyclic alkyl, a C3-C12 cycloalkyl, a C1-C10 acyclic alkenyl, C3-C12 cycloalkenyl, or a C5-C8 aryl.

Aspect 9. The compound of any one of aspects 1-8, wherein R² is further substituted with halogen, hydroxy, alkoxy, nitrile, an amine, an alkyl, an alkenyl, or an aryl.

Aspect 10. The compound of any one of aspects 1-9, wherein at least two of Z^{a}, Z^{b}, Z^{c}, Z^{d}, Z^{e}, and Z^{f} are N.

Aspect 11. The compound of any one of aspects 1-10, wherein L is a C1-C6 acyclic alkyl, a C1-C6 alkenyl, or a C5-C8 aryl.

Aspect 12. The compound of any one of aspects 1-11, wherein m is 0.

Aspect 13. The compound of any one of aspects 1-11, wherein m is 1.

Aspect 14. The compound of any one of aspects 1-13, wherein n is an integer range from 0 to 6.

Aspect 15. The compound of any one of aspects 1-13, wherein n is an integer ranging from 1 to 6.

Aspect 16. The compound of any one of aspects 1-13, wherein n is 0.

Aspect 17. The compound of any one of aspects 1-16, wherein the compound has a structure represented by a formula:

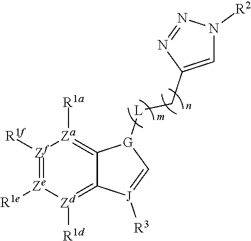

wherein R³ is hydrogen, halogen, an alkyl, or an aryl; wherein G is C or N; and wherein J is C or N.

Aspect 18. The compound of aspect 17, wherein R³ is hydrogen, halogen, a C1-C10 acyclic alkyl, or a C5-C8 aryl.

Aspect 19. The compound of aspect 17, wherein R³ is a C1-C10 acyclic alkyl or a C5-C8 aryl and wherein R³ is further substituted with halogen, hydroxy, alkoxy, nitrile, an amine, an alkyl, an alkenyl, or an aryl.

Aspect 20. The compound of any one of aspects 17-19, wherein J is N.

Aspect 21. The compound of any one of aspects 17-20, wherein G is N.

Aspect 22. A pharmaceutical composition comprising a therapeutically effective amount of a compound of any one of aspects 1-21 and a pharmaceutically acceptable carrier.

Aspect 23. A method for the treatment of a disorder associated with uncontrolled cellular proliferation in a mammal comprising the step of administering to the mammal a therapeutically effective amount of at least one compound of any one of aspects 1-21 or administering to the mammal the pharmaceutical composition of aspect 22.

Aspect 24. The method of aspect 23, wherein the mammal is a human.

Aspect 25. The method of aspect 24, wherein the mammal has been diagnosed with a need for treatment of the disorder prior to the administering step.

Aspect 26. The method of any one of aspects 23-25, further comprising the step of identifying a mammal in need of treatment of the disorder.

Aspect 27. The method of any one of aspects 23-26, wherein the disorder is associated with MHC-I dysfunction.

Aspect 28. The method of aspect 27, wherein the MHC-I dysfunction is MHC-I downregulation.

Aspect 29. The method of any one of aspects 23-28, wherein the disorder is cancer.

Aspect 30. The method of aspect 29, wherein the disorder is selected from colorectal cancer, esophageal cancer, bladder cancer, lung cancer, laryngeal cancer, breast cancer, skin cancer, and throat cancer.

Aspect 31. The method of aspect 29, wherein the disorder is selected from colorectal cancer, bladder cancer, lung cancer, laryngeal cancer, breast cancer, melanoma, and head and neck squamous cell carcinoma.

Aspect 32. A method for the upregulation of MHC-I expression in a mammal comprising the step of administering to the mammal a therapeutically effective amount of at least one compound of any of aspects 1-21 or administering to the mammal the pharmaceutical composition of aspect 22.

Aspect 33. The method of aspect 32, wherein the mammal is a human.

Aspect 34. The method of aspect 33, wherein the mammal has been diagnosed with a need for the upregulation of MHC-I expression prior to the administering step.

Aspect 35. The method of any one of aspects 32-34, further comprising the step of identifying a mammal in need of upregulation of MHC-I expression.

Aspect 36. A method for upregulation of MHC-I expression in at least one cell, comprising the step of contacting the at least one cell with an effective amount of at least one compound of any of aspects 1-21 or the pharmaceutical composition of aspect 22.

Aspect 37. The method of aspect 36, wherein the cell is mammalian.

Aspect 38. The method of aspect 36, wherein the cell is human.

Aspect 39. The method of any one of aspects 36-38, wherein the cell has been isolated from a mammal prior to the contacting step.

Aspect 40. The method of any one of aspects 36-39, wherein contacting is via administration to a mammal.

Aspect 41. The method of aspect 40, wherein the mammal has been diagnosed with a need for modulating of MHC-I expression prior to the administration.

Aspect 42. The method of aspect 40, wherein the mammal has been diagnosed with a need for treatment of a disorder related to MHC-I expression prior to the administration

Aspect 43. A kit comprising at least one compound of any of aspects 1-21 or the pharmaceutical composition of aspect 22; and one or more of: a) at least one agent known to increase MHC-I expression; b) at least one agent known to treat a disorder associated with MHC- I expression; c) at least one agent known to treat a disease of uncontrolled cellular proliferation; or d) instructions for treating a disorder associated with MHC-I dysfunction.

Aspect 44. The kit of aspect 43, wherein the at least one compound or the pharmaceutical composition and the at least one agent are co-formulated.

Aspect 45. The kit of aspect 43, wherein the at least one compound or the pharmaceutical composition and the at least one agent are co-packaged.

Aspect 46. The kit of any one of aspects 43-45, further comprising instructions to provide the compound in connection with surgery.

Aspect 47. The kit of aspect 46, wherein the instructions provide that surgery is performed prior to the administering of at least one compound.

Aspect 48. The kit of aspect 46, wherein the instructions provide that surgery is performed after the administering of at least one compound.

Aspect 49. The kit of aspect 46, wherein the instructions provide that the administering of at least one compound is to effect presurgical debulking of a tumor.

Aspect 50. The kit of aspect 46, wherein the instructions provide that surgery is performed at about the same time as the administering of at least one compound.

Aspect 51. The kit of one of aspects 43-50, further comprising instructions to provide the at least one compound or the pharmaceutical composition in connection with radiotherapy.

Aspect 52. The kit of aspect 51, wherein the instructions provide that radiotherapy is performed prior to the administering of at least one compound or the pharmaceutical composition.

Aspect 53. The kit of aspect 51, wherein the instructions provide that radiotherapy is performed after the step of the administering of at least one compound or the pharmaceutical composition.

Aspect 54. The kit of aspect 51, wherein the instructions provide that radiotherapy is performed at about the same time as the step of the administering of at least one compound or the pharmaceutical composition.

Aspect 55. The kit of one of aspects 43-54, further comprising a plurality of dosage forms, the plurality comprising one or more doses, wherein each dose comprises a therapeutically effective amount of the at least one compound or the pharmaceutical composition and the at least one agent.

Aspect 56. The kit of aspect 55, wherein each dose of the at least one compound or the pharmaceutical composition and the at least one agent are co-formulated.

Aspect 57. The kit of aspect 55, wherein each dose of the at least one compound or the pharmaceutical composition and the at least one agent are co-packaged.

Aspect 58. The kit of one of aspects 55-57, wherein the dosage forms are formulated for oral administration and/or intravenous administration.

Aspect 59. The kit of aspect 58, wherein the dosage forms are formulated for oral administration.

Aspect 60. The kit of aspect 58, wherein the dosage forms are formulated for intravenous administration.

Aspect 61. The kit of aspect 58, wherein the dosage form for the at least one compound or the pharmaceutical composition is formulated for oral administration and the dosage form for the at least one agent is formulated for intravenous administration.

Aspect 62. The kit of aspect 58, wherein the dosage form for the at least one compound or the pharmaceutical composition is formulated for intravenous administration and the dosage form for the at least one agent is formulated for oral administration.

Aspect 63. Use of a compound of at least one compound of any of aspects 1-21, in the manufacture of a medicament for the treatment of a disorder of uncontrolled cellular proliferation in a mammal.

Aspect 64. The use of aspect 63, wherein the disorder of uncontrolled cellular proliferation is associated with MHC-I dysfunction.

Aspect 65. The use of aspect 63 or aspect 64, wherein the disorder is cancer.

Aspect 66. The use of aspect 65, wherein the disorder is selected from colorectal cancer, esophageal cancer, bladder cancer, lung cancer, laryngeal cancer, breast cancer, skin cancer, and throat cancer.

Aspect 67. The use of aspect 65, wherein the disorder is selected from colorectal cancer, bladder cancer, lung cancer, laryngeal cancer, breast cancer, melanoma, and head and neck squamous cell carcinoma.

From the foregoing, it will be seen that aspects herein are well adapted to attain all the ends and objects hereinabove set forth together with other advantages which are obvious and which are inherent to the structure.

While specific elements and steps are discussed in connection to one another, it is understood that any element and/or steps provided herein is contemplated as being combinable with any other elements and/or steps regardless of explicit provision of the same while still being within the scope provided herein.

It will be understood that certain features and subcombinations are of utility and may be employed without reference to other features and subcombinations. This is contemplated by and is within the scope of the claims.

Since many possible aspects may be made without departing from the scope thereof, it is to be understood that all matter herein set forth or shown in the accompanying drawings and detailed description is to be interpreted as illustrative and not in a limiting sense.

It is also to be understood that the terminology used herein is for the purpose of describing particular aspects only, and is not intended to be limiting. The skilled artisan will recognize many variants and adaptations of the aspects described herein. These variants and adaptations are intended to be included in the teachings of this disclosure and to be encompassed by the claims herein.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### H. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Small Molecule Inducers of MHC-I Surface Expression

Introduction. The immune system can, often times, be precise, efficient, and powerful in detecting and eliminating cancerous cells. One of the principal mechanisms that the immune system leverages for cancer cell detection is through the presentation of cancer-specific peptides on the major histocompatibility complex (MHC) of the pathogenic cell.¹ In particular, MHC class I (MHC-I) is a membrane protein expressed on most nucleated cells, and is responsible for presenting short (typically 8-12 amino acids long) peptides to CD8+ T cells.² Recognition of a peptide-MHC complex (pMHC) by a CD8+ T cell through its T cell receptor (TCR) can result in a cytotoxic response through release of a host of agents including perforin and granzyme B.³ For a CD8+ T cell to be activated against a target cell (and undergo subsequent phenotypic changes), it must first recognize a ‘non-self’ peptide presented on MHC-I. The types of endogenous non-self peptides that are typically found on the surface cancer cells are broadly known as neoantigens.⁴

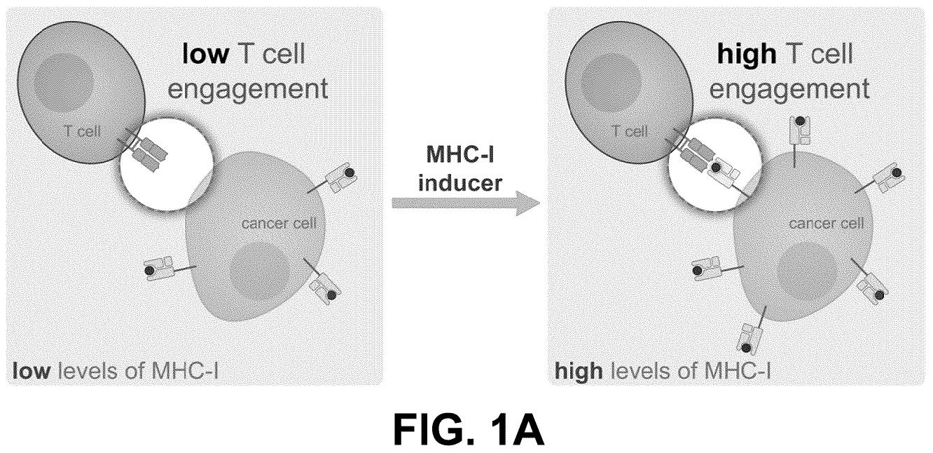

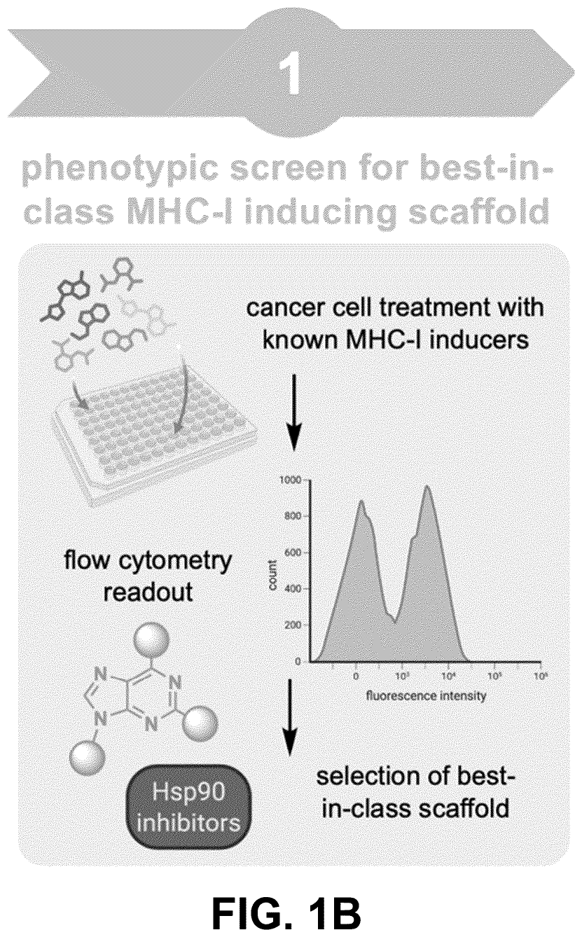

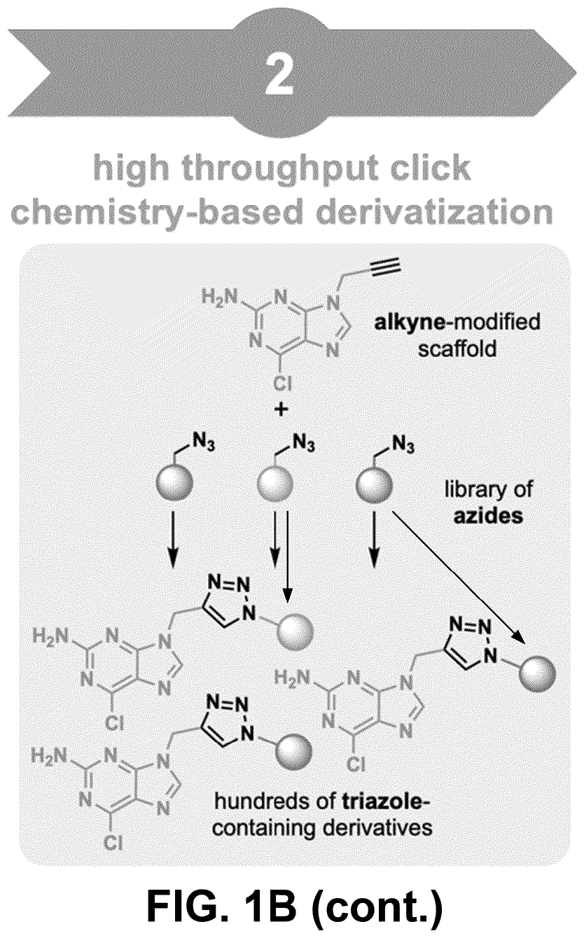

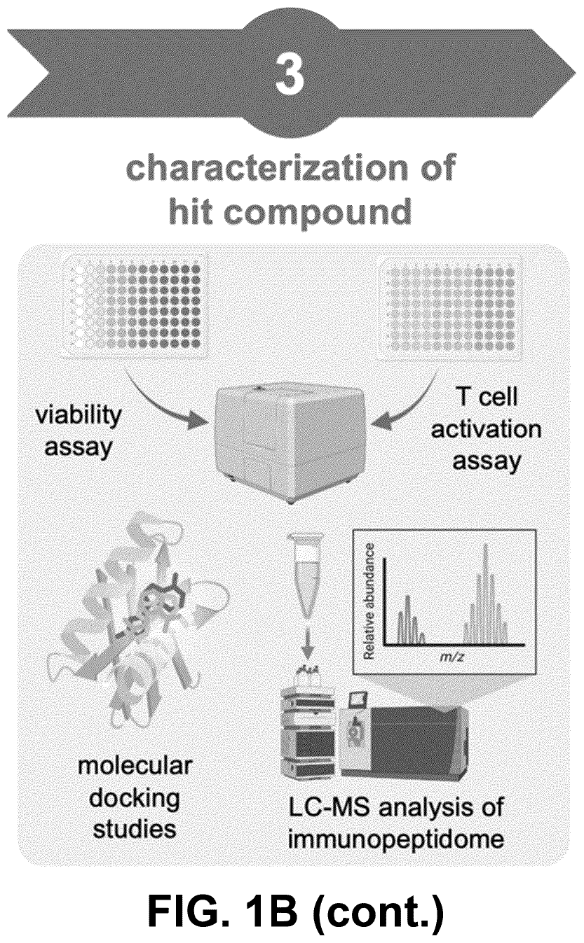

Neoantigens are generated via structural alterations to the proteome of cancer cells through amino acid substitution,⁵ post-translational modifications,⁶ and other mechanisms.⁷ These non-self peptides, which can be loaded on MHC-I for presentation, can potentially engage with TCRs on T cells.⁸ Therefore, CD8+ T cells displaying the cognate TCR are well positioned to specifically recognize and respond to neoantigen-presenting cancer cells to promote an anti-cancer immune response.⁷ In many instances, these mechanisms are central to eliminate the emergence of cancerous cells. Yet, there is considerable evidence demonstrating that cancer cells can actively avoid immune recognition by CD8+ T cells.^{9, 10} These mechanisms of resistance include, but are not limited to, remodeling of the tumor environment to be hypoxic and immunosuppressive,¹¹⁻¹³ increasing expression of immune checkpoint proteins (e.g., programmed death-ligand (PD-L1) and cytotoxic T-lymphocyte associated protein 4 (CTLA-4)),^{14, 15} promoting the secretion of immunosuppressive cytokines,¹⁶ and downregulating MHC-I molecules.¹⁷ The downregulation of surface MHC-I can directly impair patient response to programmed cell death protein 1 (PD-1) blockade immunotherapy.¹⁸ Given the tremendous success of cancer immunotherapy that directly relies on MHC-I/TCR engagement, there is a clear need to discover potent agents that promote the expression of MHC-I in cancer patients. By restoring or enhancing MHC-I expression, it may be possible to overcome immune resistance observed in many cancers, thus making the tumors more susceptible to T cell-mediated killing (FIG. 1A). Cancer cells can downregulate the expression of MHC-I as a mechanism of evasion from a patient's immunosurveillance. The use of small molecule inducers has the potential to enhance immunotherapeutic approaches that are widely used in the clinic.

Compounds that promote the expression of MHC-I span a diverse set of biological functions and include DNA methyltransferase (DMNT) inhibitors,¹⁹⁻²⁶ histone deacetylase (HDAC) inhibitors,²⁷⁻²⁹ kinase inhibitors,³⁰ heat shock protein 90 (Hsp90) inhibitors,^{31, 32} stimulator of interferon genes (STING) agonists,³³⁻³⁵ and others.³⁶⁻³⁹ Although a wider range of FDA approved agents have been screened for MHC-I induction,^{40, 41} principally, a definitive comparison across small molecule inducers has not been previously reported. In this work, a rigorous head-to-head screen of small molecules was conducted to compare their ability to enhance MHC-I surface expression in colorectal cancer cells. Additionally, Hsp90 inhibitors are shown to be able to increase the presentation of cancer-specific neoantigens. With a privileged scaffold in hand, a high-throughput diversification screen was conducted to generate a range of analogs that could be evaluated for their pharmacological properties (FIG. 1B).

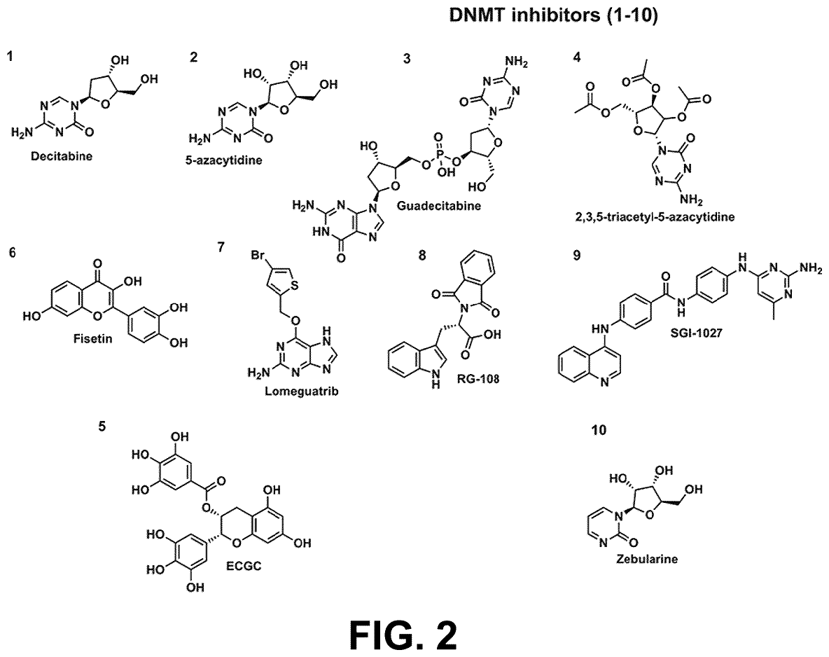

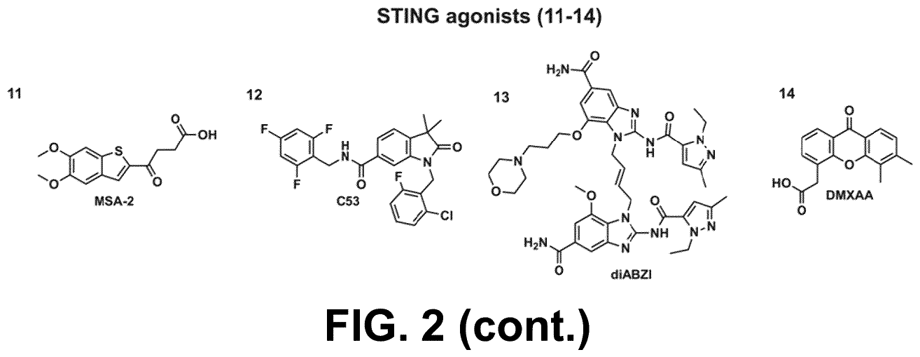

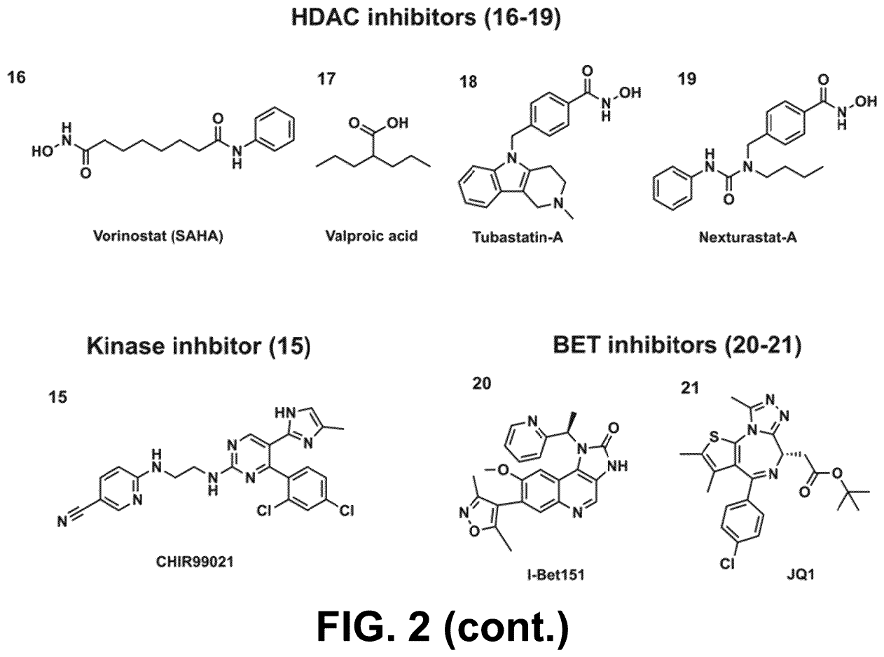

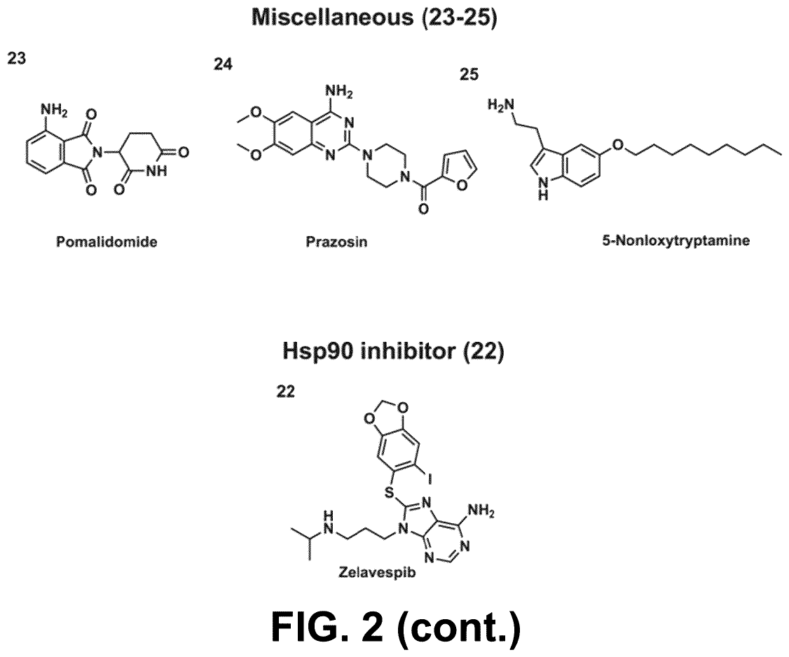

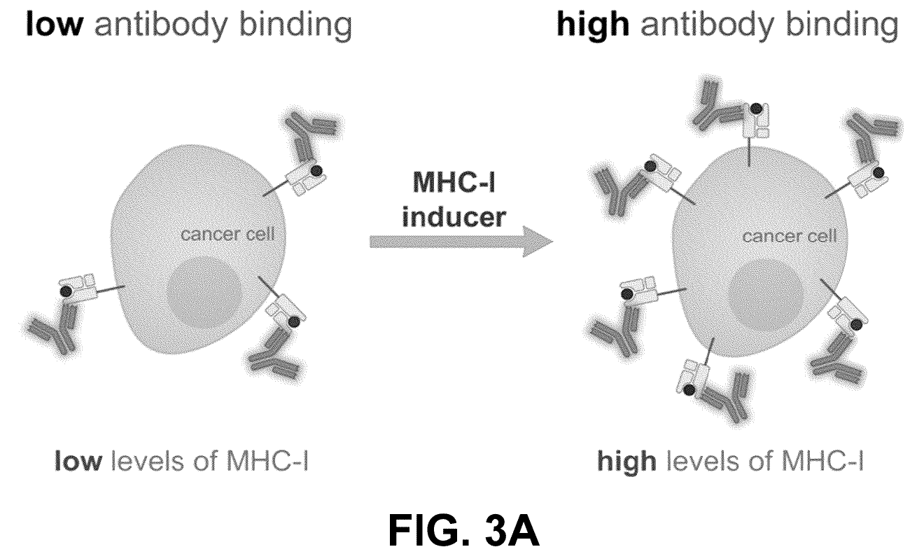

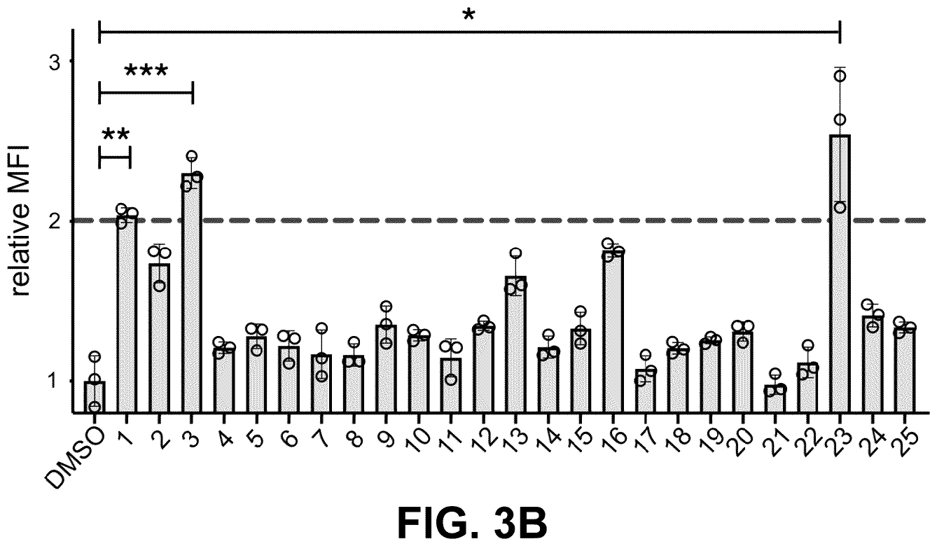

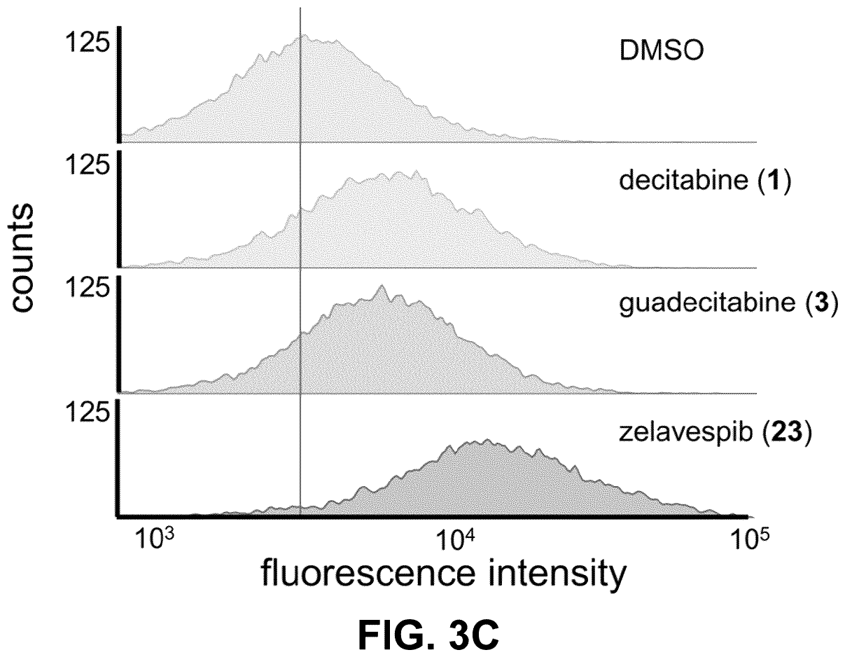

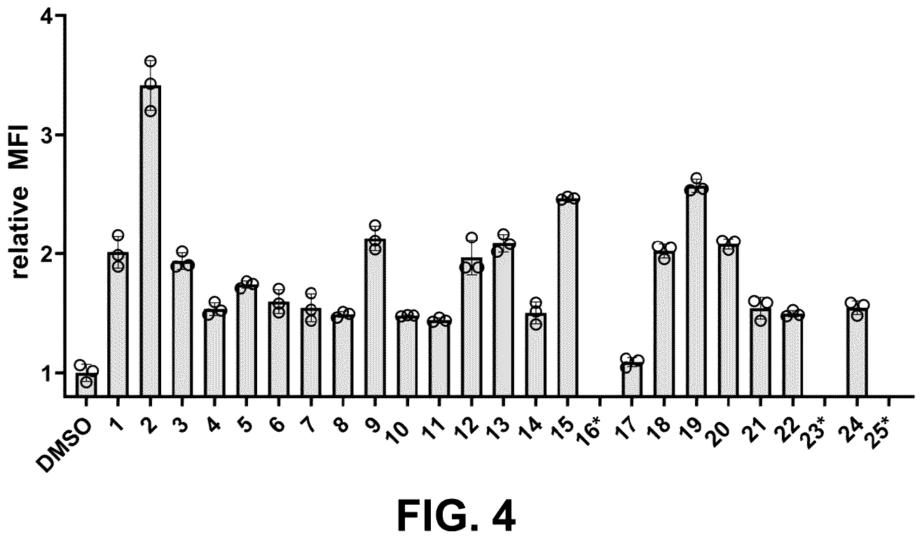

Results—Screening Small Molecules for their MHC-I Upregulation Activity. To identify the best-in-class drug scaffold for MHC-I upregulation, a flow cytometry-based assay was developed. Briefly, CT26 murine colorectal cancer cells were incubated with individual members of a library of 25 small molecules that have previously been shown to increase MHC-I expression. These included DNMT, kinase, HDAC, bromodomain and extra-terminal (BET), proteasome, and Hsp90 inhibitors, as well as STING agonists and IMiDs (FIG. 2). After cellular treatment with each individual compound, a fluorescent anti-H-2K^{d} antibody was used to quantify MHC-I expression via flow cytometry (FIG. 3A). As expected at this high concentration, most of these molecules showed an increase in MHC-I surface expression at a concentration of 5 mM (FIG. 4). Still, it was notable that some of the molecules did not show any enhancement above background. To identify the most potent inducers of MHC-I surface expression, a second screen was performed at a more stringent concentration of 1 mM. Results showed that three of the small molecules exhibited an increase in MHC-I surface expression above a two-fold cutoff (FIG. 3B). The identified MHC-I enhancers were primarily in two pharmacological classes: DNMT inhibitors (decitabine 1 and guadecitabine 3) and Hsp90 inhibitors (zelavespib 23). After incubation of the three lead compounds at a concentration of 500 nM with CT26 cells, it was found that 23 led to higher overall expression of MHC-I on the surface compared to the two DNMT inhibitors (FIG. 3C).

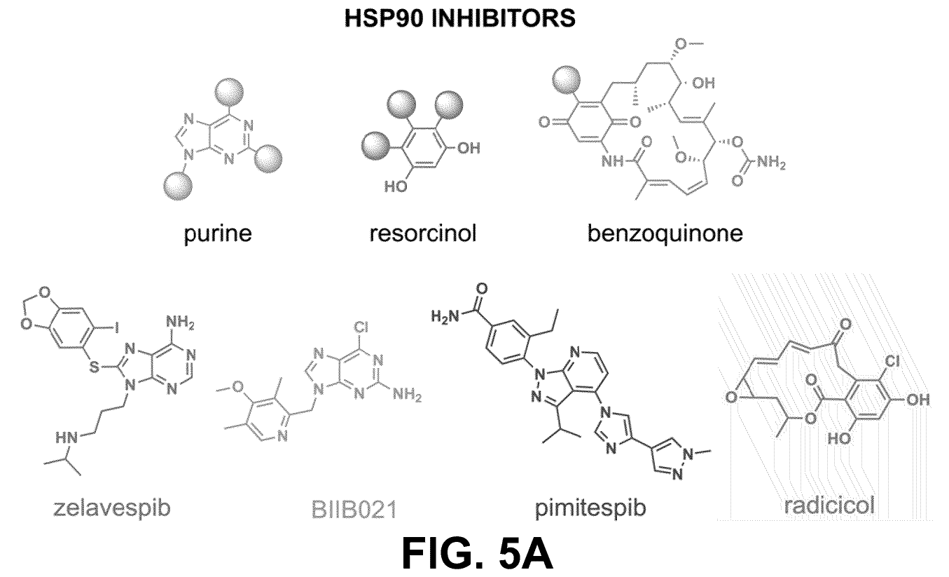

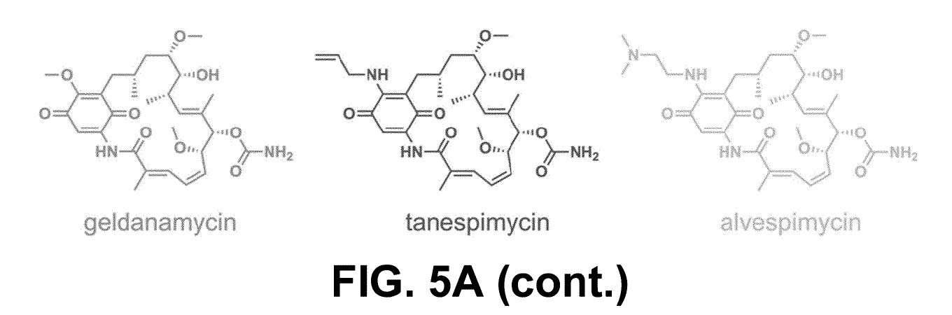

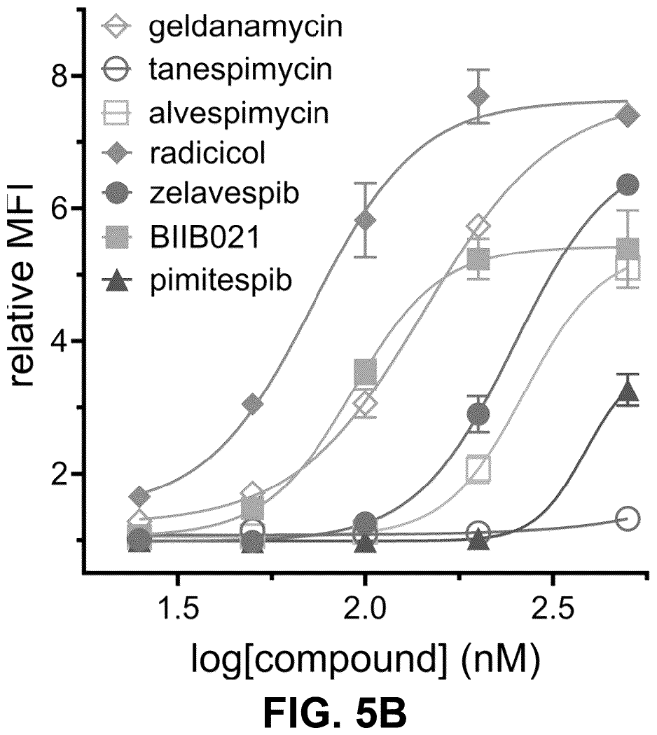

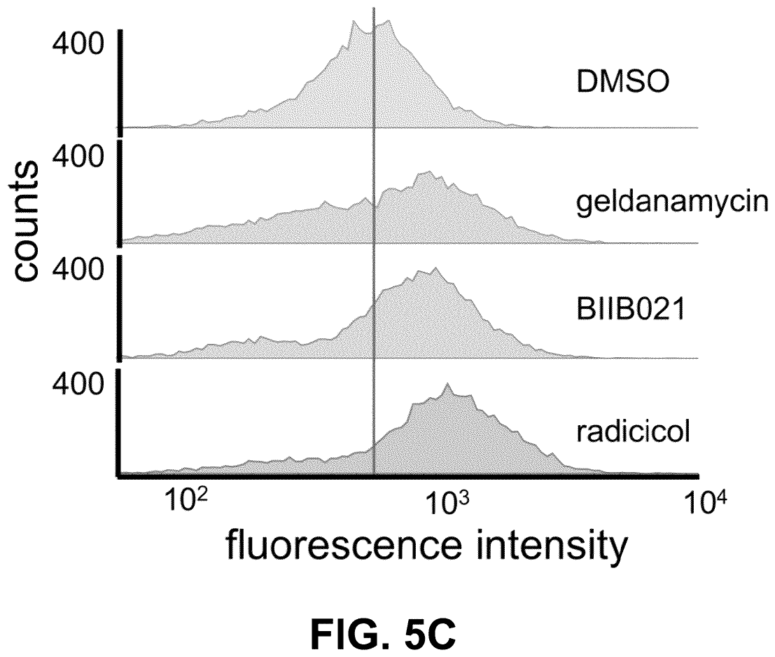

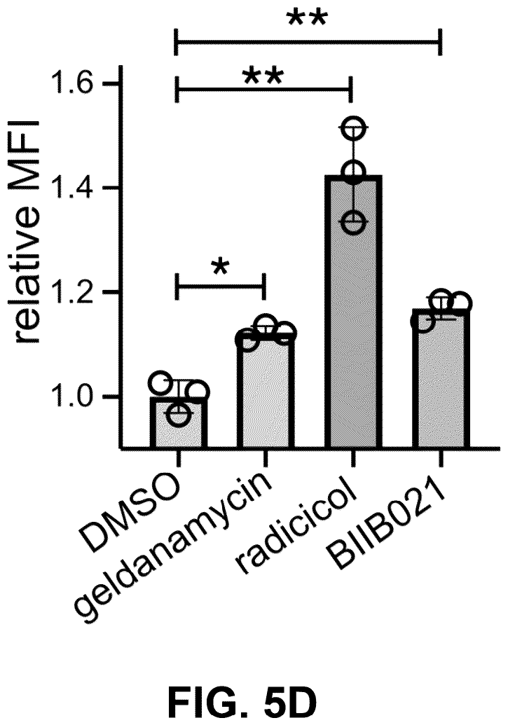

Results—Screening Hsp90 Inhibitors for MHC-I Surface Upregulation Activity. To further explore the relationship between Hsp90 inhibitors and MHC-I surface expression, six additional Hsp90 inhibitors were examined (FIG. 5A). In total within this sub-library, three of molecules were from the purine-based family, one from the resorcinol family, and three from the benzoquinone family of Hsp90 inhibitors. To more readily assess their potency, a concentration scan was performed in CT26 cells rather than a single concentration analysis. Results revealed that all but two (pimitespib and tanespimycin) of the Hsp90 inhibitors tested had EC₅₀ values in the nanomolar range for MHC-I surface expression. Among these, it was found that radicicol, BIIB021 and geldanamycin had the lowest EC₅₀ values, at 72±1, 92±1, and 14435 1 nM, respectively. Interestingly, together these top hits covered all three primary classes of Hsp90 inhibitors; this could potentially suggest that Hsp90 inhibition is a primary driver of the phenotypic observation of MHC-I induction. Presumably, if any off-target activity were to be observed and if it were to be the driver of the induction, there is likely to be a single class that is favored. The top hits enhanced MHC-I surface expression in CT26 cells by approximately 5 to 8-fold compared to basal expression levels, a marked increase relative to the initial hit (zelavespib 23) that prompted the focus on Hsp90 inhibitors (FIG. 5B). To ensure that Hsp90 inhibitors could operate in other cellular contexts, they were tested in the human colorectal cancer cell line HCT116 and found to be effective MHC-I inducers (FIG. 5C). Overall, these results demonstrate that Hsp90 inhibitors are potent inducers of MHC-I surface expression, and this prompted further exploration to assemble a larger and more diverse structure activity relationship campaign.

In theory, the enhancement of MHC-I surface expression should broadly sample a greater breadth of cytosolic peptides including potential neoantigens. This is key because the efficacy of checkpoint blockage therapy relies principally on cytotoxic CD8+ T cells recognizing neoantigens presented on cancer cell surfaces. Next, the potential upregulation of specific antigens from live cells upon their treatment with Hsp90 inhibitors was investigated (FIG. 5D). To test this, murine MC38-OVA cells were used, which are genetically modified to express the protein ovalbumin (OVA).⁴² OVA contains the sequence SIINFEKL, which has been previously used as a model neoantigen. Upon the intracellular processing of OVA and the production of SIINFEKL, it is known that this peptide can be presented by H-2K^{b} and is recognized by SIINFEKL-specific CD8+ T cells.⁴³ MC38-OVA cells were treated with 100 nM of Hsp90 inhibitors for 48 hours, followed by incubation with a fluorescent antibody specific for H-2K^{b} bound to SIINFEKL. Satisfyingly, cellular treatment with Hsp90 inhibitors led to a significant increase in the presentation of the model neoantigen SIINFEKL, indicating that Hsp90 inhibitors can potentially promote the presentation of neoantigen-specific pMHC complexes (FIG. 5D).

Results—High-Throughput Click Chemistry Diversification Strategy of Hsp90 Inhibitor.

With the three top candidates in hand, the next step was to further diversify a core scaffold to broadly understand how structure could potentially drive MHC-I upregulation. A large-scale sub-library around a single agent could provide a larger set of agents that can be tested and selected for specific biological properties (e.g., improved toxicity profile, solubility, and selectivity). From the three agents, BIIB021 was moved forward with due to its potency (with many similar structures in clinical evaluation for Hsp90 inhibition)⁴⁴⁻⁴⁶ and its robust chemical structure. Given the nature of the derivatization strategy, it was important to consider the potential stability of the starting scaffold; both radicicol and geldanamycin have structural fragments that are known to have low inherent chemical stability. Also, the availability of the crystal structure of BIIB021 in complex with Hsp90 can provide an avenue to understand how the analogs might interact with their target protein.⁴⁷

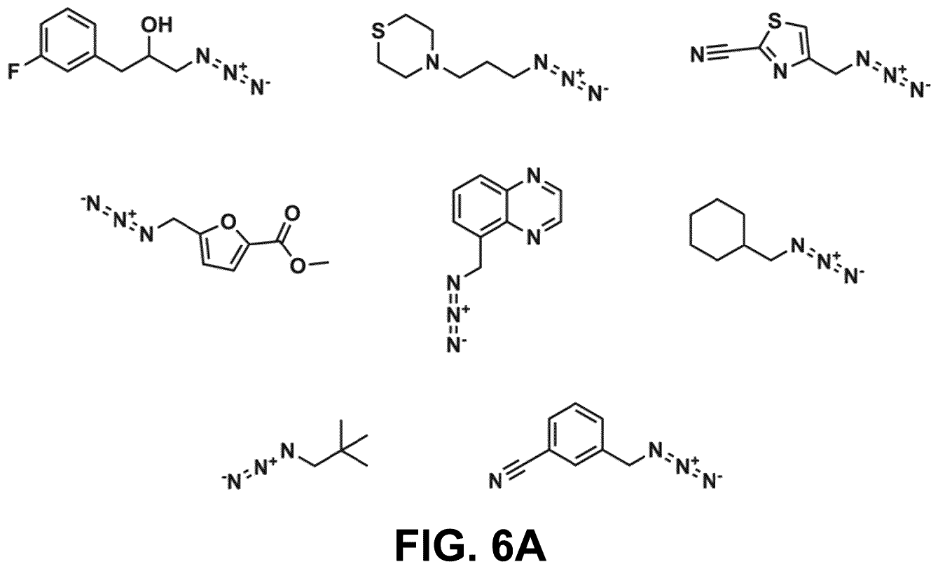

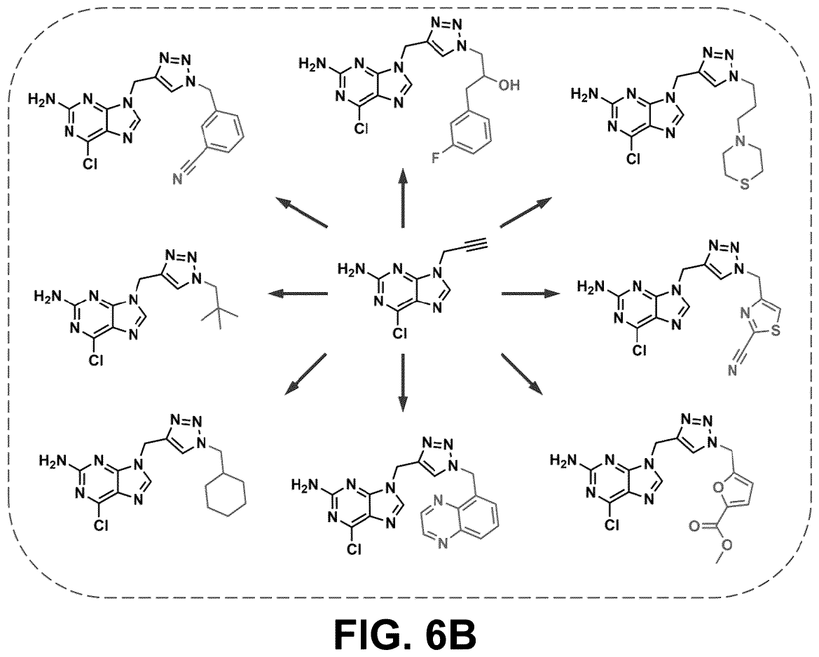

For the generation of the library, in situ click chemistry was used. In this format, an alkyne is installed within the core scaffold, and this parent molecule is plated into a microwell plate system with each well containing a new azide-tagged fragment. This approach presents considerable advantages for drug discovery of MHC-I inducers. Click reactions have a high level of specificity and efficiency, particularly exemplified by the Cu(I)-catalyzed azide-alkyne cycloaddition (CuAAC), which enables precise modifications and synthesizes complex molecules with minimal byproducts.⁴⁸⁻⁵⁰ The versatility inherent in using a library of azides allows for the exploration of diverse molecular combinations and structural variations, essential for identifying drug candidates with optimal pharmacological properties. Moreover, it facilitates the simultaneous screening and synthesis of potential drug candidates, speeding up the identification of active compounds. Recently, this click chemistry-based strategy has been used to identify small molecule modulators of glucagon-like-peptide-1 receptor.⁵¹ It has also been shown with this method that over 80% of azide molecules formed triazole products at yields of 70% or higher.⁵² See FIG. 6A and FIG. 6B for example azide compounds and triazole products.

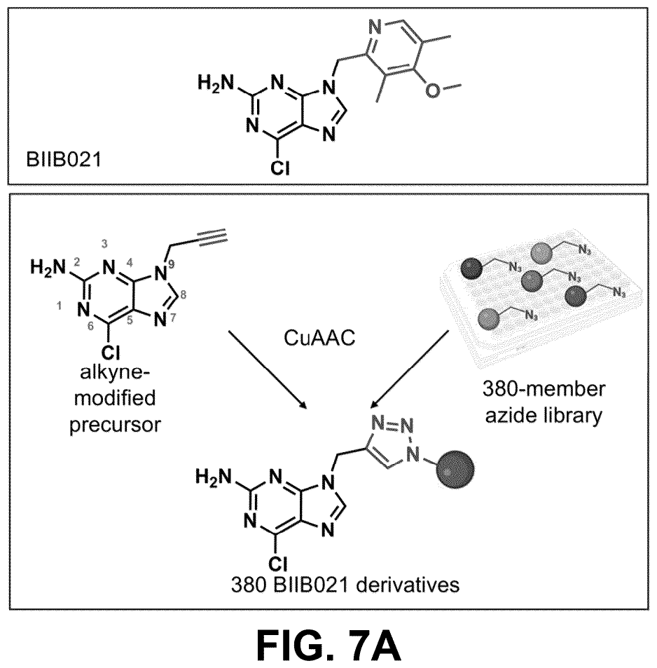

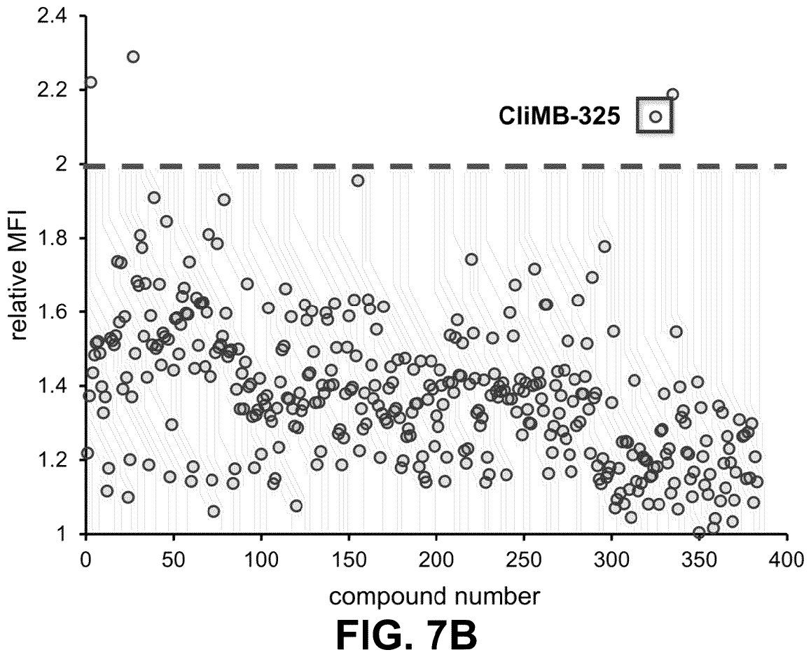

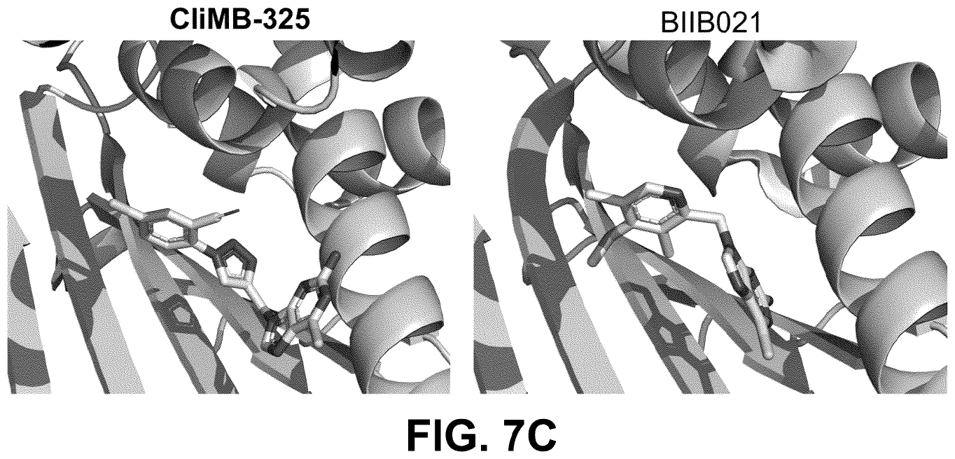

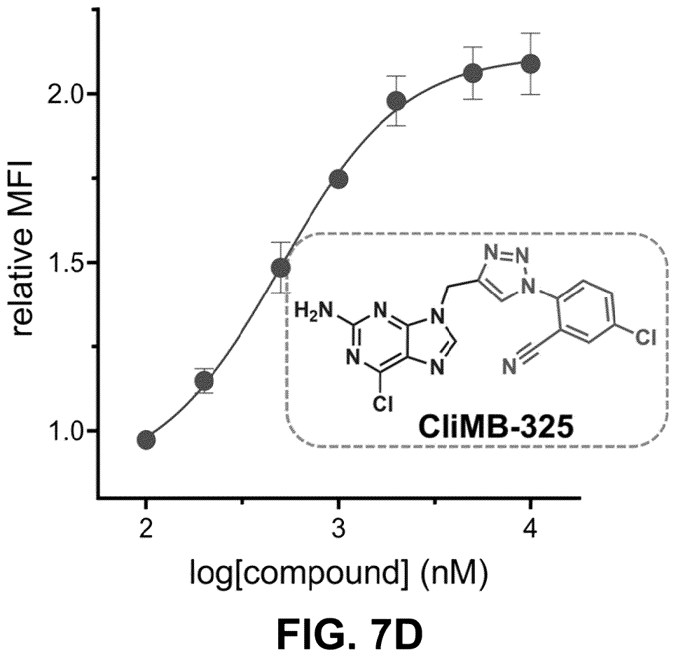

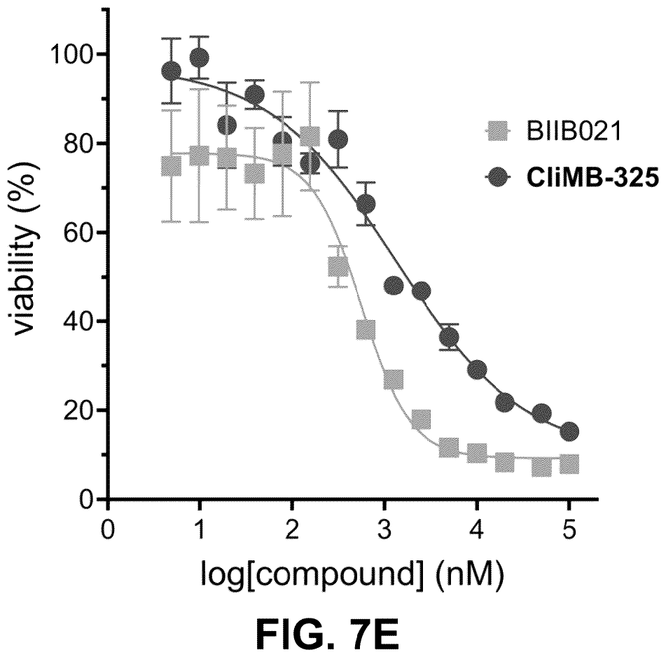

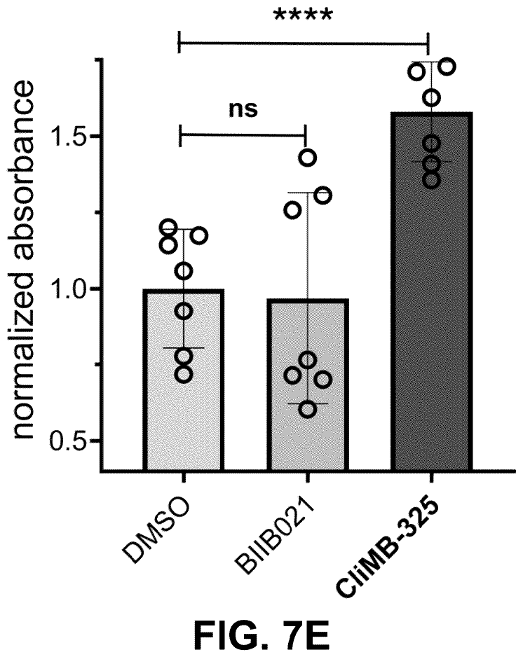

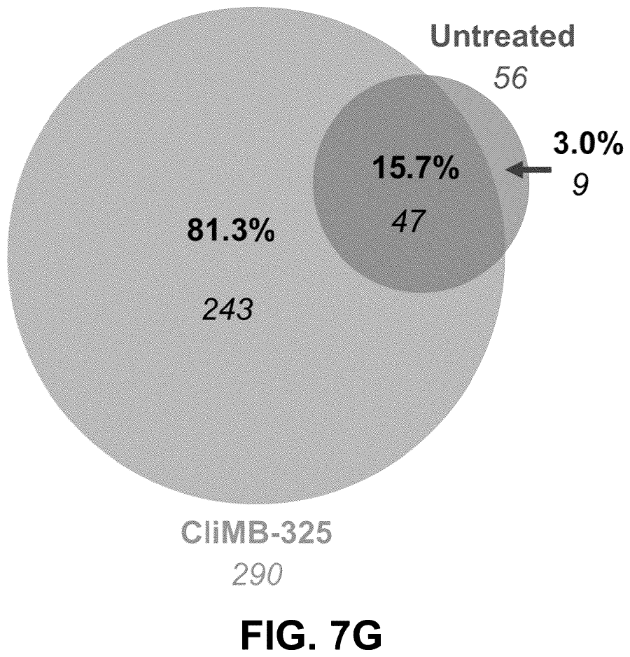

In the context of the core purine scaffold, a site to install the alkyne handle should be considered. From the crystal structure of BIIB021 in complex with Hsp90, the N9 position of the purine core was identified as a solvent-exposed site that is amendable to chemical modification.⁴⁷ Moreover, previous reports have demonstrated that this position can be leveraged to access purine-based analogs while retaining Hsp90 inhibition.⁵³ Therefore, the envisioned approach involved modifying a precursor of BIIB021, 2-amino-6-chloropurine, with an alkyne on the N9 position in order to react it with a library of small molecule azides (FIG. 7A). It was reasoned that the resulting triazole ring from the click reaction would structurally mimic the pyridine ring of BIIB021 and allow rapid generation of hundreds of derivatives.

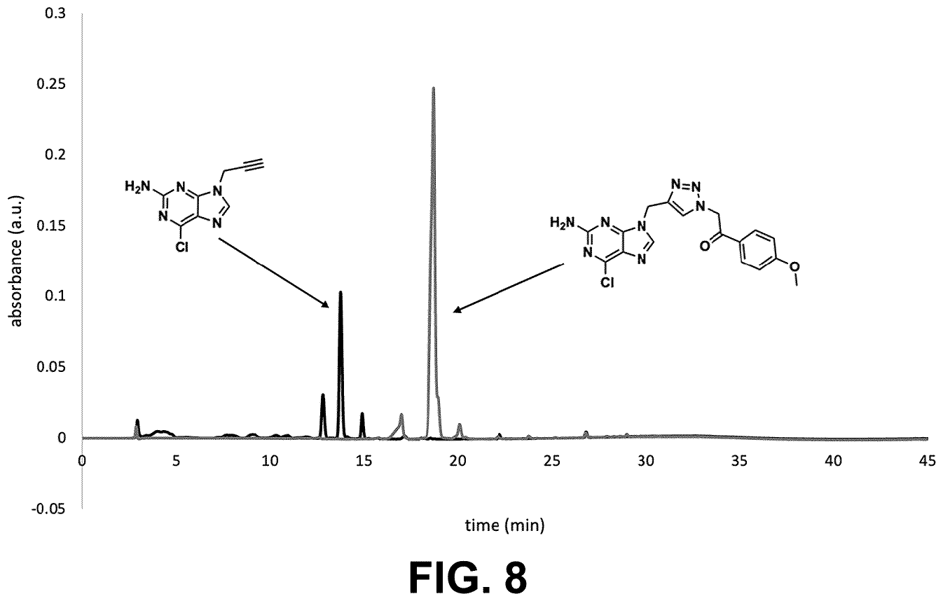

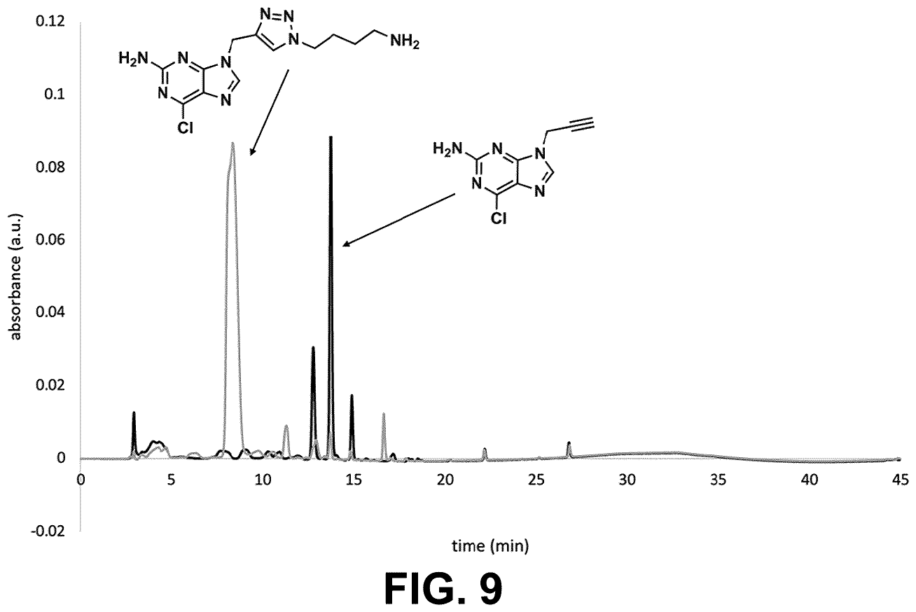

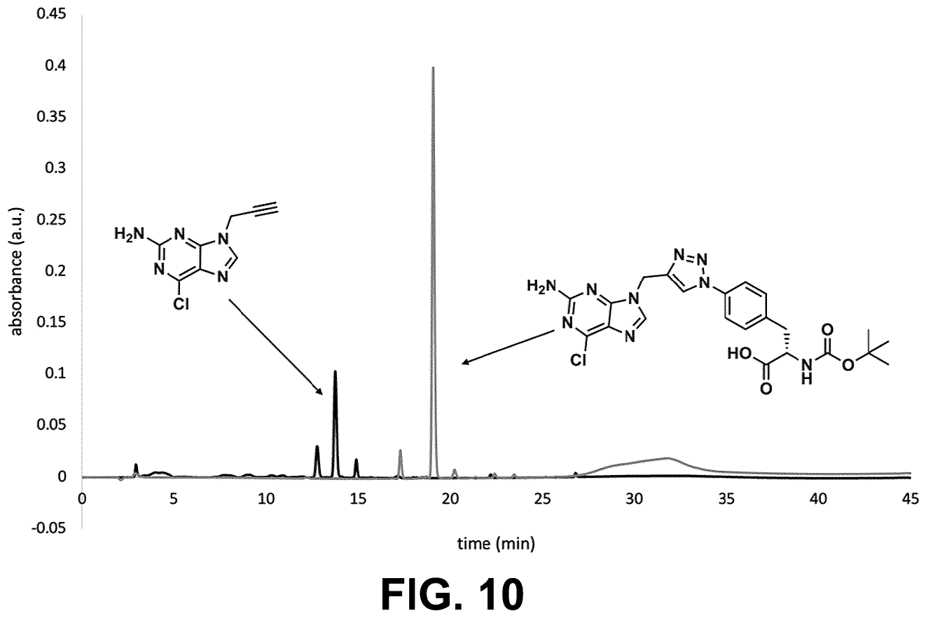

To build the alkyne-bearing purine, 2-amino-6-chloropurine was reacted with propargyl bromide to yield the major product 9-propargyl-2-amino-6-chloropurine. The other product was the N7 regioisomer, which was separated during purification. The identity of isolated N9 alkyne-bearing compound was confirmed by NMR and its purity was analyzed by RP-HPLC. Next, a model in situ CuAAC test reaction was performed with the alkyne-modified precursor and a small subset of azide bearing molecules. To cover the potential variability in types of chemical structures found in the full library, a subset of molecules were selected that varied in size, polarity, and steric environment surrounding the azide. Of note, the reactions were performed in microwell plates in an experimental procedure that mimicked the conditions for the eventual in situ set of reactions. All three reactions showed a conversion rate of >90% to the triazole product (FIG. 8-10). Regarding FIG. 8, 9-propargyl-2-amino-6-chloropurine and 2-azido-1-(4-methoxy-phenyl)-ethanone, each at a concentration of 10 mM, were reacted with 40 mM L-ascorbic acid and 2 mM CuSO₄/THPTA in a 3:2 ratio of DMSO to water, with a total reaction volume of 100 μL. Regarding FIG. 9, 9-propargyl-2-amino-6-chloropurine and 4-azido-1-butanamine, each at a concentration of 10 mM, were reacted with 40 mM L-ascorbic acid and 2 mM CuSO4/THPTA in a 3:2 ratio of DMSO to water, with a total reaction volume of 100 μL. Regarding FIG. 10, 9-propargyl-2-amino-6-chloropurine and Boc-4-azido-L-phenylalanine, each at a concentration of 10 mM, were reacted with 40 mM L-ascorbic acid and 2 mM CuSO4/THPTA in a 3:2 ratio of DMSO to water, with a total reaction volume of 100 μL. With the model reactions showing high levels of conversion, the reaction conditions were well-suited for a larger screen. The goal of utilizing a larger library was to ensure that modifications to the purine core would cover a large chemical space to broadly sample the engagement with the target. Moreover, given the nature of the phenotypic assay, it can be reasoned that the diversity of this library could also be useful in improving other properties that are necessary for a lead candidate including high accumulation levels, low off-target effects, and reduced toxicity.

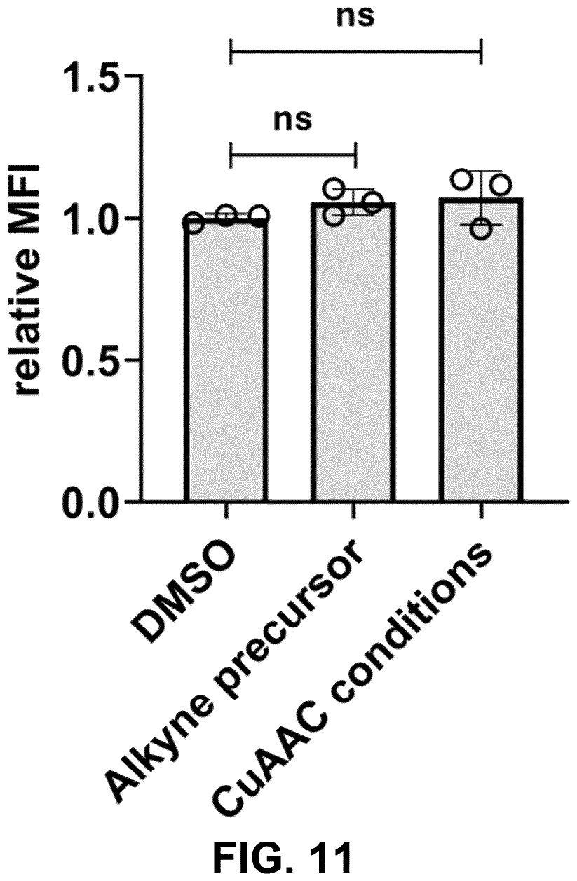

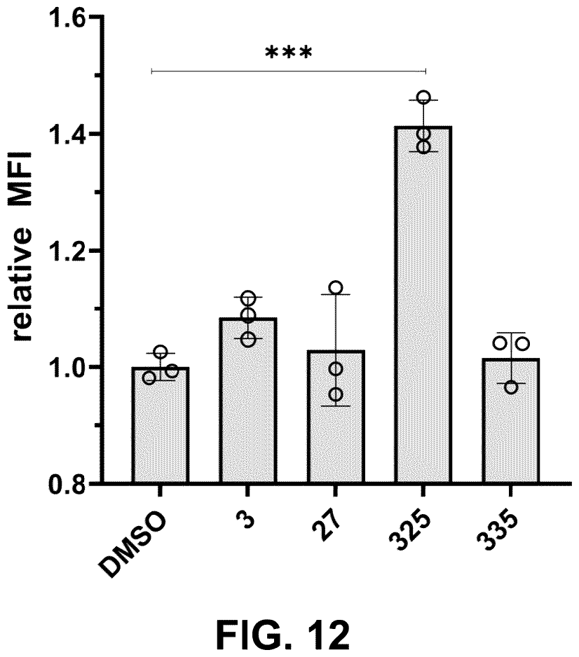

In total, 380 azide-containing small molecules were dispensed into wells containing the alkyne-bearing purine analog and the click reaction reagents. After the reaction step, contents of each well were incubated with CT26 cells, and MHC-I induction was monitored by treatment with a fluorescently tagged antibody as previously described. Critically, incubation of CT26 cells with the alkyne precursor alone or the additional click reagents did not result in any increase in MHC-I surface expression, confirming that the reaction reagents themselves have no inherent activity (FIG. 11). This indicates that the reaction reagents have no activity on their own. The results from the 380-member screen revealed that four of the click reaction mixtures (3, 27, 325, and 335; structures shown below), showed an increase in MHC-I surface expression above a two-fold cutoff over the DMSO control (FIG. 7B). To further confirm these results, cells were treated with each of four reaction mixtures in a more stringent concentration (theoretically 500 nM, assuming complete conversion). From these results, cell treatment with compound 325 led to the highest levels of MHC-I surface expression (FIG. 12). The click product between the alkyne precursor and compound 325 of the azide library was then synthesized and purified to yield ‘Click MHC-I Booster-325’ (CliMB-325).

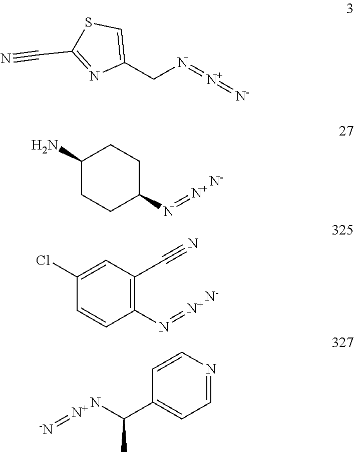

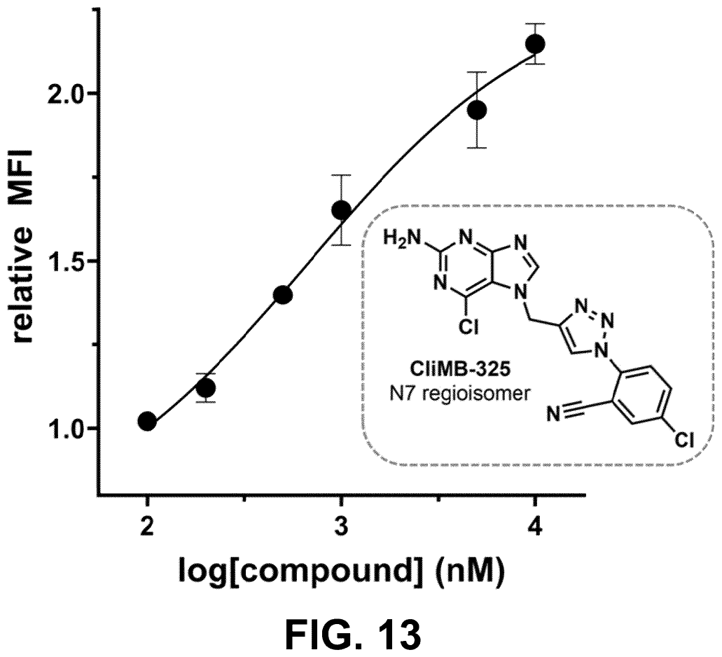

Results-CliMB-325 Enhances MHC-I Surface Expression and T Cell Activation. Finally, the purine-based lead agent CliMB-325 was further characterized. By docking CliMB-325 into Hsp90 using RosettaLigand, CliMB-325 was found to bind in a similar manner to BIIB021 (PDB ID: 3qdd) with no measurable deviation in Hsp90 conformation (FIG. 7C). Next, to establish its potency, a concentration scan of CliMB-325 was performed using CT26 cells to assess its ability to increase MHC-I expression. Results showed that CliMB-325 retained MHC-I upregulation activity with an EC₅₀ of 498 nM (FIG. 7D). Notably, the N7 position of the purine core in BIIB021 has also been shown as a site that tolerates chemical modifications while retaining Hsp90 inhibition activity.⁴⁵ As such, azide 325 was reacted with the minor product of the alkyne-modified precursor (7-propargyl-2-amino-6-chloropurine) to synthesize the N7 isomer of CliMB-325. This product was found to exhibit comparable levels of MHC-I upregulation activity to CliMB-325 in CT26 cells (FIG. 13). Given the failed clinical progression of BIIB021 due to toxicity issues,⁴⁴ MTT cell viability assay was also performed comparing BIIB021 with CliMB-325. The results indicated that CliMB-325 demonstrated a 2.4-fold improvement in toxicity over its parent compound, BIIB021, with CC₅₀ values of 1.3±0.0 nM μM and 563±1 nM, respectively (FIG. 7E).

Given that the downregulation of MHC-I surface expression reduces T cell recognition of neoantigens, whether the upregulation of MHC-I induced by CliMB-325 results in enhanced T cell activation was investigated. To do so, the B3Z T cell hybridoma cell line was used, which contains OVA-specific TCRs and expresses the enzyme β-galactosidase under the control of an IL-2 inducible promoter. Upon B3Z recognition of the OVA-pMHC complex on OVA expressing cells, the subsequent IL-2 production promotes the expression of β-galactosidase. b-galactosidase activity can then be measured via hydrolysis of the reagent chlorophenol red-β-galactopyranoside (CPRG) which leads to a color change that is reflective of T cell activation levels.⁵⁴ MC38-OVA cells were incubated with 100 nm BIIB021 and CliMB-325 for 48 hours before being co-cultured with B3Z T cells for six hours. β-galactosidase expression was then measured via the colorimetric reagent CPRG on a plate reader at 570 nm. While TCR activation in cells treated with BIIB021 did not significantly differ from the DMSO control, MC38-OVA cells treated with CliMB-325 exhibited a nearly 1.6-fold increase in TCR activation levels (FIG. 7F).

Data are represented as mean±SD (n=7).

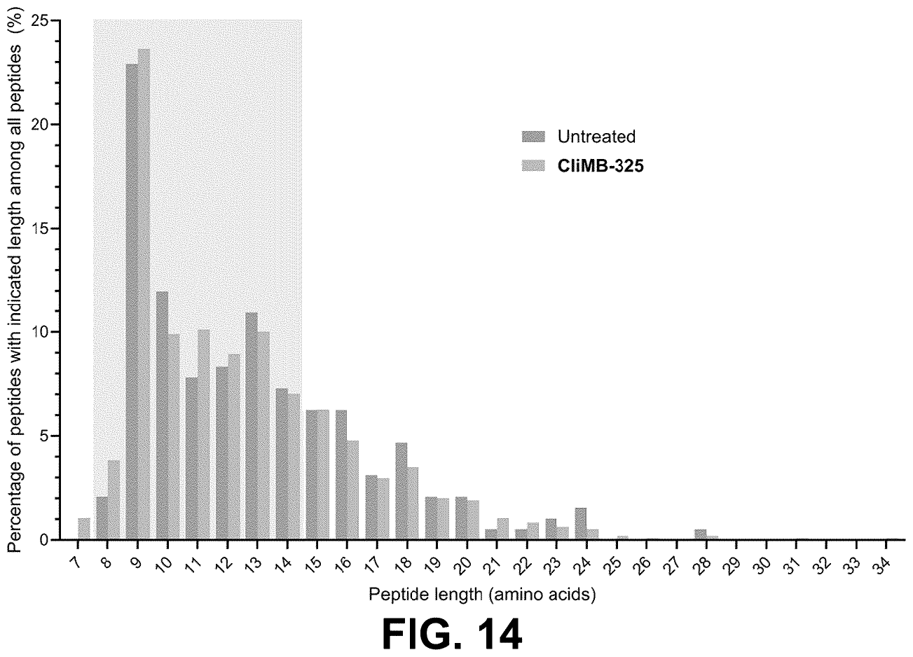

Building upon the finding that CliMB-325 increases MHC-I surface expression in CT26 cells, potential changes to the immunopeptidome were investigated, including the enhancement of peptides presented on the cell surface. To this end, CT26 cells were incubated with or without CliMB-325 for 48 hours, and MHC-I-bound peptides were isolated via mild acid elution (MAE) and sequenced using mass spectrometry.⁶⁷ After removing known contaminants, the resulting peptide list was filtered to include only those with the optimal length (8-14 amino acids) for MHC-I binding, which were found to constitute the majority of identified peptides (FIG. 14).⁶⁸ These peptides were further refined based on NetMHCpan 4.1 prediction scores to retain only putative binders of one or more of the three MHC allotypes expressed by the CT26 cells. Strikingly, CliMB-325 treatment resulted in a 5-fold increase in the number of unique peptide sequences presented by MHC-I compared to untreated cells (FIG. 7G). Among the 290 MHC-I peptides identified in the CliMB-325-treated sample, 81.3% were exclusive to this group and absent in untreated cells. Moreover, of the 56 total MHC-I peptides identified in the untreated group, 84% were also present in the CliMB-325-treated sample. This high degree of overlap in the immunopeptidome between the untreated and CliMB-325-treated samples suggests that CliMB-325 treatment enhances the detection of peptides present in the existing immunopeptidome. Interestingly, the observed upregulation of surface MHC-I with CliMB-325 treatment was modest (1.5-2-fold) compared to the impact on the immunopeptidome (5-fold), which may reflect a threshold effect in antigen presentation. While the concentration of peptides is generally the rate-limiting factor in the antigen presentation response, the relationship between MHC-I abundance and peptide diversity is nonlinear.² Under conditions of limited MHC-I availability, peptide binding is highly competitive and high-affinity peptides dominate the repertoire. In this scenario, even a modest increase in available MHC-I can greatly (and disproportionately) expand the range of peptides successfully presented. Overall, these results validate the use of a high-throughput click chemistry screening approach to generate bioactive compounds with MHC-I upregulation activity.

Materials—Reagents. All library compounds were purchased from either Selleck Chemicals, AK Scientific, A2B Chem, MedChemExpress, GlpBio, Cayman Chemical Company, or AmBeed. Compounds were solubilized in DMSO and stored at −20° C. Recombinant murine and human IFN-γ were purchased from PeproTech. APC-labeled antibodies against H-2K^{d}/H-2D^{d}, HLA-A, B,C, and H-2K^{b} bound to SIINFEKL were purchased from BioLegend. The library of 380 azide-containing small molecules were purchased from Enamine (catalog # AZD-380-X-100). For the synthesis of CliMB-325, 2-amino-6-chloropurine was purchased from AmBeed (catalog # A135577) and 2-azido-5-chlorobenzonitrile was purchased from Enamine (catalog #EN300-279694). Dulbecco's Modified Eagle's Medium (DMEM), Roswell Park Memorial Institute (RPMI) 1640 medium, and McCoy's 5A medium were purchased from VWR. Fetal Bovine Serum (FBS) and penicillin-streptomycin were purchased from Sigma-Aldrich.

Experimental Methods—Mammalian Cell Culture. CT26 cells were cultured in RPMI 1640 media supplemented with 10% fetal bovine serum, 50 IU/mL penicillin, and 50 μg/mL streptomycin. HCT116 cells were kindly provided by Dr. Anja-Katrin Bielinsky and were cultured in McCoy's 5A media supplemented with 10% fetal bovine serum, 50 IU/mL penicillin, 50 μg/mL streptomycin, and 2 mM GlutaMAX. MC38-OVA cells were kindly provided by Dr. Mirna Perusina Lanfranca and were cultured in DMEM supplemented with 10% fetal bovine serum, 50 IU/mL penicillin, 50 μg/mL streptomycin, 50 μg/mL gentamycin, and 10 μg/mL blasticidin. B3Z cells were kindly provided by Dr. Aaron Esser-Kahn and maintained in RPMI 1640 media supplemented with 10% fetal bovine serum, 50 IU/mL penicillin, and 50 ug/mL streptomycin. All cells were cultured in T75 flasks and maintained in a humidified atmosphere of 5% CO2 at 37° C.

Experimental Methods—Flow Cytometry-Based Assays. 1.5×10⁴ cells were seeded in a treated 96-well plate along with indicated concentrations of library compounds at 37° C. After 48 hours, cells were washed once with PBS, removed using TrypLE™ Express Enzyme (Thermo Fisher), and transferred to a round-bottom 96-well plate. Transferred cells were centrifuged (1100×g, 5 min) in a Thermo Scientific Jouan C4i centrifuge, and the cell pellets were resuspended and fixed in 4% formaldehyde solution for 20 minutes. The plate was centrifuged (1100×g, 5 min) and pelleted cells were resuspended in a 1:100 dilution of indicated fluorescence antibodies in culture media for 1 hour at 4° C. Flow cytometry was performed using the following antibodies: APC anti-mouse H-2K^{d}/H-2D^{d} (clone 34-1-2S), APC anti-human HLA-A,B,C (clone W6/32), or APC anti-mouse H-2K^{b} bound to SIINFEKL (clone 25-D1.16). Cells were analyzed using an Attune NxT Flow Cytometer (Thermo Fisher) equipped with a 637 nm laser with 670/14 nm bandpass filter.

Experimental Methods—MTT Cell Viability Assay. 1.5×10⁴ CT26 cells were seeded in a treated 96-well plate, either with or without compounds (BIIB021 and CliMB-325) at indicated concentrations at 37° C. After 48 hours, a solution of MTT in PBS (filter sterilized through a 0.2-μM filter) was added to each well to achieve a final concentration of 0.45 mg/mL. After incubating at 37° C. for 2 hours, cells were centrifuged (1100×g, 5 min) in a Thermo Scientific Jouan C4i centrifuge and the supernatant was removed. 100 μL of DMSO was added to each well to dissolve the formation of formazan precipitate. The absorbance of the solution in each well was read at 570 nm using a BioTek Synergy H1 Microplate Reader. Wells containing no cells (only the added DMSO) were used as a negative control for viability, while untreated cells served as the positive control for 100% viability.

Experimental Methods—B3Z T Cell Activation. 1.5×10⁴ MC38-OVA cells were seeded in a treated 96 well plate, either with or without compounds (BIIB021 and CliMB-325) at indicatedd concentrations at 37° C. After 48 hours, the culture media was replaced with media containing 10⁵ B3Z cells, which were co-incubated with the MC38-OVA cells for 6 hours. Cells were centrifuged (1100×g, 5 min) in a Thermo Scientific Jouan C4i centrifuge and the supernatant was removed. Lysis buffer containing 0.2% saponin, 500 mM CPRG reagent, 20 mM MgCl_{2,} and 100 mM β-mercaptoethanol in 1X PBS was added to each well. After 45 minutes, absorbance at 570 nm was recorded using a BioTek Synergy H1 Microplate Reader.

Experimental Methods—Molecular Docking Studies. Conformational predictions of CliMB-325 in Hsp90 were performed using RosettaLigand using the crystal structure of BIIB021 bound to Hsp90 (PDB ID: 3qdd).⁶¹⁻⁶⁴ Native crystal structure was prepared for docking by removing all water molecules and co-crystallized ligands. PyMOL was used for visualization of the docking results.

Experimental Methods—Mild Acid Elution (MAE) of MHC-I-Bound Peptides. The MAE protocol was adapted from a previously published protocol.⁶⁷ MAEs from CT26 cells were performed with 1×10⁷ CT26 cells per sample. Cells were washed three times with PBS then were treated for 90 seconds with MAE buffer. The MAE buffer consisted of 131 mM citric acid, 66 mM Na₂HPO₄, and 150 mM NaCI adjusted to pH 3.3 with NaOH. Following treatment with the MAE buffer, the eluted peptide solution was centrifuged (4000×g, 5 min) in a Thermo Scientific Jouan C4i centrifuge. The peptide-containing supernatant was collected and acidified with 0.1% TFA. The obtained peptide solution was further purified on Oasis HLB columns (barrel size 1 cm³, 30 mg of sorbent; Waters, product no.: WAT094225) prerinsed with 100% acetonitrile (MeCN)/0.1% TFA. After equilibration with 100% H₂O/0.1% TFA, sample loading, and washing with 95% MeCN/0.1% TFA, peptides were eluted with 60% MeCN/0.1% TFA. The eluate was filtered through a 15 mL Amicon ultrafilter device with 3 kDa molecular weight cutoff (Merck Millipore, Cat.-No. UFC901024) then lyophilized to dryness using a Labconco Freezone 4.5 L (−84° C.) lyophilizer.

Experimental Methods—Liquid Chromatography. Peptide separation was carried out on a Vanquish Neo UHPLC system using a trap-and-elute setup. An Aurora Frontier™ TS C18 column (IonOpticks; 60 cm×75 μm, 1.7 μm particles) was used for peptide separation. The mobile phases used were: Phase A—0.1% formic acid (FA) in water; Phase B—80% acetonitrile (ACN) with 0.1% FA in water. A 40-minute gradient was applied at 0.3 μl/min: 15% to 50% MPB from 0.1 to 40 min, followed by 50% to 99% mobile phase buffer B (MPB) from 40.1 to 42 min, held at 99% MPB until 50 min, and re-equilibrated at 1% MPB for 35 minutes.

Experimental Methods—Mass Spectrometry Data Acquisition. Data were acquired using an Orbitrap Astral mass spectrometer in a DDA mode. For MS1 scans, the Orbitrap resolution was set at 120,000 with an AGC target of 100%. MS1 spectra were recorded over an m/z range of 350-1350, with a maximum injection time of 50 ms. The isolation window for MS2 precursor selection was set to 1.2 m/z. Up to 30 scans were acquired per MS1 cycle for precursor ions with intensities greater than 5.0×10³ and charge states ranging from 2 to 6. MS2 fragmentation was performed with HCD at 25% collision energy and a maximum injection time of 25 ms. Dynamic exclusion was turned on with a duration of 20 seconds.
Experimental Methods—DDA Data Analysis. Spectra were converted to mzXML using a modified version of ReAdW.exe. Mass spectra were processed using a COMET-based software pipeline and searched against the mouse UniProt database (downloaded on Oct. 3, 2024). Database searches were performed using a precursor ion tolerance of 50-ppm and 0.02 Da fragment ion tolerance. No enzyme specificity was specified for peptide identification. Carbamidomethylation of cysteine residues (+57.021 Da) were set as static modifications, while oxidation of methionine residues (+15.995 Da) was set as a variable modification. Peptide-spectrum matches (PSMs) were adjusted to a 1% false discovery rate (FDR) using standard target-decoy approaches.⁶⁹ Sequenced peptides were further filtered based on length (8-14 amino acids) to ensure likelihood of MHC-I binding. The obtained peptide list was input into NetMHCpan 4.1 database to predict binding compatibility with the MHC-I allotypes expressed by CT26 cells (H-2-Dd, H-2-K^{d}, and H-2-Ld).⁷⁰ Prediction score was reported by NetMHCpan 4.1, and downstream peptide analysis was only performed on peptides which were modeled to bind with a prediction score of 2.0 or lower and thus expected to be putative binders of MHC-I molecules.
Experimental Methods—Synthesis of Target Compounds. NMR spectroscopy was performed in DMSO-d₆ on a Varian 600 MHz spectrophotometer. Chemical shifts are reported in ppm (δ) and coupling constants (J) are reported in Hertz [Hz]. High-resolution electrospray ionization mass spectrometry (HR-ESI-MS) was performed using an Agilent 1260 Infinity II Prime liquid chromatography (LC) system. Compounds were analyzed for purity using RP analytical HPLC equipped with Waters 1525 with a 2489 UV/Visible Detector monitoring at 311 nm wavelength, on a Phenomenex Luna 5 μM C18 (2) 250×4.6 mm column using gradient elution. Flow rate=1.0 mL/min; mobile phase A=0.1% trifluoroacetic acid (TFA) (v/v) in water; mobile phase B=0.1% TFA in methanol (MeOH); gradient: 5% B for 5 min, 5-100% B for 20 min, 100% B hold for 3 min, then 100-5% B for 15 min. All compounds are >95% pure by HPLC analysis

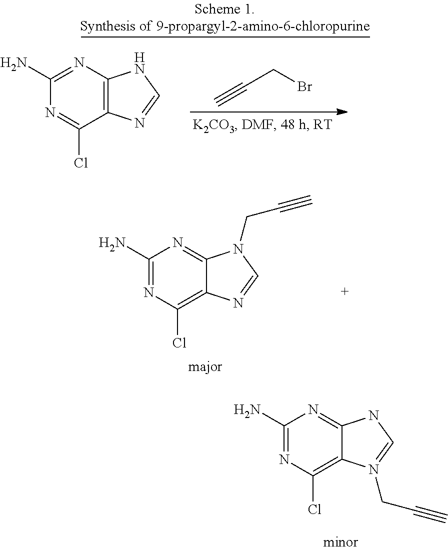

9-propargyl-2-amino-6-chloropurine was synthesized based on literature procedure.⁶⁵ 2-amino-6-chloropurine (3.0 g, 1 eq) was suspended DMF (50 mL) followed by addition of anhydrous K₂CO₃ (2.934 g, 1.2 eq) and stirring under N₂ atmosphere for 1 hour. After this time, propargyl bromide (1.894 g, 0.9 eq) was added and stirred for 48 hours under N₂ atmosphere at room temperature. DMF was evaporated at 60° C. under high vacuum to afford a yellowish-white powder. A 1:2 ratio of minor and major compound was produced as determined by NMR. The crude material was purified by reverse-phase preparative high-performance liquid chromatography (RP-HPLC) equipped with Waters 1525 with a 2489 UV/Visible Detector monitoring at 311 nm wavelength, on a Phenomenex Luna Omega 5 μM Polar C18 250×21.2 mm column using gradient elution with using H₂O/MeOH with 0.1% TFA at 10 mL/min. The HPLC fractions of the major compound were concentrated under reduced pressure using a rotary evaporator, then lyophilized to dryness using a Labconco Freezone 4.5 L (−84° C.) lyophilizer and characterized by NMR which matched with the reported compound.⁶⁶ This product was analyzed for purity using RP analytical HPLC equipped with Waters 1525 with a 2489 UV/Visible Detector monitoring at 311 nm wavelength, on a Phenomenex Luna 5 μM C18 (2) 250×mm column using gradient elution with using H₂O/MeOH with 0.1% TFA at 1 mL/min. The major product was used for click chemistry. White solid; 66 1H NMR (600 MHZ, DMSO-d₆) d 8.18 (s, 1H, 8-H), 7.02 (brs, 2H, −NH₂), 4.93 (d, 2H, J=1 Hz, —CH₂), 3.48 (t, 1H, J=1 Hz, C≡CH). HRMS: m/z calculated for C₈H₆CIN5 [M+H]⁺208.0385, found 208.0388.

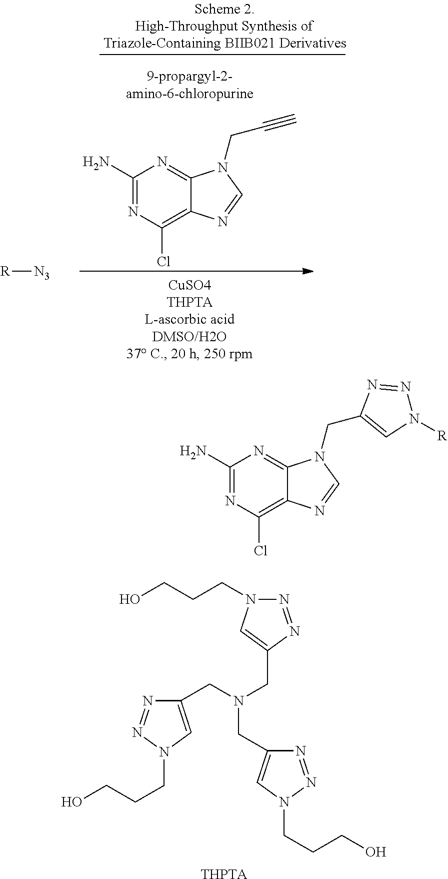

Triazole analogs were synthesized based on literature procedure.₅₁ Azide solutions from the azide library in Plates 1-5 were initially at a concentration of 100 mM in DMSO. Azides were added in each well of a 96-well plate at a concentration of 10 mM. To each well of this newly loaded plate, L-ascorbic acid solution was added to a concentration of 40 mM along with 10 mM of 9-propargyl-2-amino-6-chloropurine (synthesis shown in Scheme 1) and 2 mM of CuSO_{4/}THPTA in a solution of DMSO and water at a 3:2 ratio to a total volume of 100 μL. The plates were sealed and swirled at 250 rpm and 37° C. for 20 hours to afford the corresponding triazole product in each well.

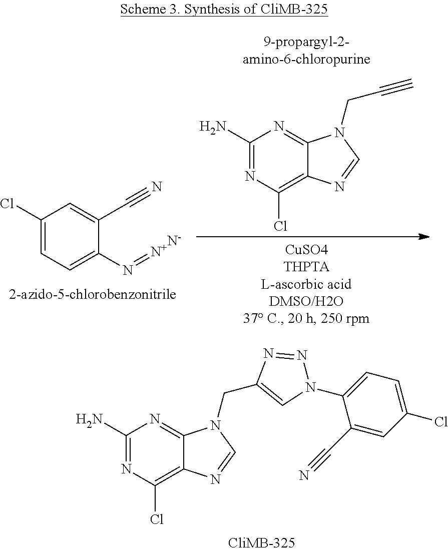

In a 50 ml conical tube, the following reagents were added: 10 mM of 2-azido-5-chlorobenzonitrile (azide #325 from 380 screen), 40 mM of aqueous L-ascorbic acid, 10 mM of 9-propargyl-2-amino-6-chloropurine (synthesis shown in Scheme 1), 2 mM of aqueous CuSO₄/THPTA solution, in a 3:2 ratio of DMSO to water at a total volume of 15 mL. The tube was swirled at 250 rpm and 37° C. for 20 hours to yield CliMB-325. The compound was purified by reverse-phase preparative high-performance liquid chromatography (RP-HPLC) equipped with Waters 1525 with a 2489 UV/Visible Detector monitoring at 311 nm wavelength, on a Phenomenex Luna Omega 5 μM Polar C18 250×21.2 mm column using gradient elution with using H2O/MeCN with 0.1% TFA at 10 mL/min. The HPLC fractions of the desired purified product were concentrated under reduced pressure using a rotary evaporator, then lyophilized to dryness using a Labconco Freezone 4.5 L (−84° C.) lyophilizer. The product was analyzed for purity using RP analytical HPLC equipped with Waters 1525 with a 2489 UV/Visible Detector monitoring at 311 nm wavelength, on a Phenomenex Luna 5 μM C18 (2) 250×mm column using gradient elution with using H2O/MeCN with 0.1% TFA at 1 mL/min. The final product was stored at −20° C. until further use, and stocks were made at 10 mM in DMSO. White solid; 1H NMR (600 MHZ, DMSO-d₆) d 8.73 (s, 1H, 8-H), 8.36 (d, 1H, J=1 Hz, Ar—H), 8.24 (s, 1H, triazole-H), 8.04 (dd, 1H, J=3.6 Hz, Ar-H), 7.88 (d, 1H, J=3.6 Hz, Ar—H), 6.95 (brs, 2H, —NH), 5.51 (s, 2H, —CH2); 13C NMR (150 MHz, DMSO-d₆) d 159.9, 153.9, 149.4, 143.4, 142.9, 136.5, 134.7, 134.5, 134.1, 127.3, 124.7, 123.2, 114. 6, 108.6, 38.0. HRMS: m/z calculated for C₁₅H₁₀Cl₂N₉ [M+H]⁺ 386.0431, found 386.0439.

Conclusion. Here, compounds that have immunomodulatory effects on colorectal cancer cell lines have been identified. Hsp90 inhibitors were found to be among the most potent class of molecules tested that increase MHC-I surface expression as well as promote the display of cancer-specific neoantigens for CD8+ T cell recognition. Leveraging an Hsp90 inhibitor core scaffold, also demonstrated as proof-of-concept is a high-throughput click chemistry-based screening platform for the discovery of molecules with immunomodulatory activity.

### I. REFERENCES

References are cited herein throughout using the format of superscripted reference number(s) corresponding to one or more of the following numbered references. For example, citation of references numbers 1 and 2 immediately herein below would be indicated in the disclosure as^{1,2}.

Ref. 1 Dersh, D., Holly, J., and Yewdell, J. W. (2021) A few good peptides: MHC class I-based cancer immunosurveillance and immunoevasion, Nat Rev Immunol 21, 116-128.

Ref. 2 Neefjes, J., Jongsma, M. L., Paul, P., and Bakke, O. (2011) Towards a systems understanding of MHC class I and MHC class II antigen presentation, Nat Rev Immunol 11, 823-836.

Ref. 3 Huppa, J. B., and Davis, M. M. (2003) T-cell-antigen recognition and the immunological synapse, Nat Rev Immunol 3, 973-983.

Ref. 4 Boon, T., Van Pel, A., De Plaen, E., Chomez, P., Lurquin, C., Szikora, J. P., Sibille, C., Mariame, B., Van den Eynde, B., Lethe, B., and et al. (1989) Genes coding for T-cell-defined tum transplantation antigens: point mutations, antigenic peptides, and subgenic expression, Cold Spring Harb Symp Quant Biol 54 Pt 1, 587-596.

Ref. 5 Mardis, E. R. (2019) Neoantigens and genome instability: impact on immunogenomic phenotypes and immunotherapy response, Genome Med 11, 71.

Ref. 6 Kacen, A., Javitt, A., Kramer, M. P., Morgenstern, D., Tsaban, T., Shmueli, M. D., Teo, G. C., da Veiga Leprevost, F., Barnea, E., Yu, F., Admon, A., Eisenbach, L., Samuels, Y., Schueler-Furman, O., Levin, Y., Nesvizhskii, A. I., and Merbl, Y. (2023) Post-translational modifications reshape the antigenic landscape of the MHC I immunopeptidome in tumors, Nat Biotechnol 41, 239-251.

Ref. 7 Xie, N., Shen, G., Gao, W., Huang, Z., Huang, C., and Fu, L. (2023) Neoantigens: promising targets for cancer therapy, Signal Transduct Target Ther 8, 9.

Ref. 8 Kelly, J. J., Bloodworth, N., Shao, Q., Shabanowitz, J., Hunt, D., Meiler, J., and Pires, M. M. (2024) A Chemical Approach to Assess the Impact of Post-translational Modification on MHC Peptide Binding and Effector Cell Engagement, ACS Chem Biol 19, 1991-2001.

Ref. 9 Roerden, M., and Spranger, S. (2025) Cancer immune evasion, immunoediting and intratumour heterogeneity, Nat Rev Immunol.

Ref. 10 Galassi, C., Chan, T. A., Vitale, I., and Galluzzi, L. (2024) The hallmarks of cancer immune evasion, Cancer Cell 42, 1825-1863.

Ref. 11 Fu, Z., Mowday, A. M., Smaill, J. B., Hermans, I. F., and Patterson, A. V. (2021) Tumour Hypoxia-Mediated Immunosuppression: Mechanisms and Therapeutic Approaches to Improve Cancer Immunotherapy, Cells 10.

Ref. 12 Warburg, O. (1956) On the origin of cancer cells, Science 123, 309-314.

Ref. 13 Li, Y., Patel, S. P., Roszik, J., and Qin, Y. (2018) Hypoxia-Driven Immunosuppressive Metabolites in the Tumor Microenvironment: New Approaches for Combinational Immunotherapy, Front Immunol 9, 1591.

Ref. 14 He, X., and Xu, C. (2020) Immune checkpoint signaling and cancer immunotherapy, Cell Res 30, 660-669.

Ref. 15 Dong, H., Strome, S. E., Salomao, D. R., Tamura, H., Hirano, F., Flies, D. B., Roche, P. C., Lu, J., Zhu, G., Tamada, K., Lennon, V. A., Celis, E., and Chen, L. (2002) Tumor-associated B7-H1 promotes T-cell apoptosis: a potential mechanism of immune evasion, Nat Med 8, 793-800.

Ref. 16 Tie, Y., Tang, F., Wei, Y. Q., and Wei, X. W. (2022) Immunosuppressive

cells in cancer: mechanisms and potential therapeutic targets, J Hematol Oncol 15, 61.

Ref. 17 Taylor, B. C., and Balko, J. M. (2022) Mechanisms of MHC-I Downregulation and Role in Immunotherapy Response, Front Immunol 13, 844866.

Ref. 18 Zaretsky, J. M., Garcia-Diaz, A., Shin, D. S., Escuin-Ordinas, H., Hugo, W., Hu-Lieskovan, S., Torrejon, D. Y., Abril-Rodriguez, G., Sandoval, S., Barthly, L., Saco, J., Homet Moreno, B., Mezzadra, R., Chmielowski, B., Ruchalski, K., Shintaku, I. P., Sanchez, P. J., Puig-Saus, C., Cherry, G., Seja, E., Kong, X., Pang, J., Berent-Maoz, B., Comin-Anduix, B., Graeber, T. G., Tumeh, P. C., Schumacher, T. N., Lo, R. S., and Ribas, A. (2016) Mutations Associated with Acquired Resistance to PD-1 Blockade in Melanoma, N Engl J Med 375, 819-829.

Ref. 19 Russo, S., Feola, S., Feodoroff, M., Chiaro, J., Antignani, G., Fusciello, M., D'Alessio, F., Hamdan, F., Pellinen, T., Molsa, R., Tripodi, L., Pastore, L., Gronholm, M., and Cerullo, V. (2024) Low-dose decitabine enhances the efficacy of viral cancer vaccines for immunotherapy, Mol Ther Oncol 32, 200766.

Ref. 20 Fonsatti, E., Nicolay, H. J., Sigalotti, L., Calabro, L., Pezzani, L., Colizzi, F., Altomonte, M., Guidoboni, M., Marincola, F. M., and Maio, M. (2007) Functional up-regulation of human leukocyte antigen class I antigens expression by 5-aza-2′-deoxycytidine in cutaneous melanoma: immunotherapeutic implications, Clin Cancer Res 13, 3333-3338.

Ref. 21 Terracina, K. P., Graham, L. J., Payne, K. K., Manjili, M. H., Baek, A., Damle, S. R., and Bear, H. D. (2016) DNA methyltransferase inhibition increases efficacy of adoptive cellular immunotherapy of murine breast cancer, Cancer Immunol Immunother 65, 1061-1073.

Ref. 22 Ma, R., Rei, M., Woodhouse, I., Ferris, K., Kirschner, S., Chandran, A., Gileadi, U., Chen, J. L., Pereira Pinho, M., Ariosa-Morejon, Y., Kriaucionis, S., Ternette, N., Koohy, H., Ansorge, O., Ogg, G., Plaha, P., and Cerundolo, V. (2022) Decitabine increases neoantigen and cancer testis antigen expression to enhance T-cell-mediated toxicity against glioblastoma, Neuro Oncol 24, 2093-2106.

Ref. 23 Krishnadas, D. K., Bao, L., Bai, F., Chencheri, S. C., and Lucas, K. (2014) Decitabine facilitates immune recognition of sarcoma cells by upregulating CT antigens, MHC molecules, and ICAM-1, Tumour Biol 35, 5753-5762.

Ref. 24 Son, C. H., Lee, H. R., Koh, E. K., Shin, D. Y., Bae, J. H., Yang, K., and Park, Y. S. (2016) Combination treatment with decitabine and ionizing radiation enhances tumor cells susceptibility of T cells, Sci Rep 6, 32470.

Ref. 25 Pappalardi, M. B., Keenan, K., Cockerill, M., Kellner, W. A., Stowell, A., Sherk, C., Wong, K., Pathuri, S., Briand, J., Steidel, M., Chapman, P., Groy, A., Wiseman, A. K., McHugh, C. F., Campobasso, N., Graves, A. P., Fairweather, E., Werner, T., Raoof, A., Butlin, R. J., Rueda, L., Horton, J. R., Fosbenner, D. T., Zhang, C., Handler, J. L., Muliaditan, M., Mebrahtu, M., Jaworski, J. P., McNulty, D. E., Burt, C., Eberl, H. C., Taylor, A. N., Ho, T., Merrihew, S., Foley, S. W., Rutkowska, A., Li, M., Romeril, S. P., Goldberg, K., Zhang, X., Kershaw, C. S., Bantscheff, M., Jurewicz, A. J., Minthorn, E., Grandi, P., Patel, M., Benowitz, A. B., Mohammad, H. P., Gilmartin, A. G., Prinjha, R. K., Ogilvie, D., Carpenter, C., Heerding, D., Baylin, S. B., Jones, P. A., Cheng, X., King, B. W., Luengo, J. I., Jordan, A. M., Waddell, I., Kruger, R. G., and McCabe, M. T. (2021) Discovery of a first-in-class reversible DNMT1-selective inhibitor with improved tolerability and efficacy in acute myeloid leukemia, Nat Cancer 2, 1002-1017.

Ref. 26 Coral, S., Sigalotti, L., Gasparollo, A., Cattarossi, I., Visintin, A., Cattelan, A., Altomonte, M., and Maio, M. (1999) Prolonged upregulation of the expression of HLA class I antigens and costimulatory molecules on melanoma cells treated with 5-aza-2′-deoxycytidine (5-AZA-CdR), J Immunother 22, 16-24.

Ref. 27 Mora-Garcia Mde, L., Duenas-Gonzalez, A., Hernandez-Montes, J., De la Cruz-Hernandez, E., Perez-Cardenas, E., Weiss-Steider, B., Santiago-Osorio, E., Ortiz-Navarrete, V. F., Rosales, V. H., Cantu, D., Lizano-Soberon, M., Rojo-Aguilar, M. P., and Monroy-Garcia, A. (2006) Up-regulation of HLA class-I antigen expression and antigen-specific CTL response in cervical cancer cells by the demethylating agent hydralazine and the histone deacetylase inhibitor valproic acid, J Transl Med 4, 55.

Ref. 28 Woan, K. V., Lienlaf, M., Perez-Villaroel, P., Lee, C., Cheng, F., Knox, T., Woods, D. M., Barrios, K., Powers, J., Sahakian, E., Wang, H. W., Canales, J., Marante, D., Smalley, K. S. M., Bergman, J., Seto, E., Kozikowski, A., Pinilla-Ibarz, J., Sarnaik, A., Celis, E., Weber, J., Sotomayor, E. M., and Villagra, A. (2015) Targeting histone deacetylase 6 mediates a dual anti-melanoma effect: Enhanced antitumor immunity and impaired cell proliferation, Mol Oncol 9, 1447-1457.

Ref. 29 Narukawa, T., Yasuda, S., Horinaka, M., Taniguchi, K., Tsujikawa, T., Morita, M., Ukimura, O., and Sakai, T. (2024) The Novel HDAC Inhibitor OBP-801 Promotes MHC Class I Presentation Through LMP2 Upregulation, Enhancing the PD-1-Targeting Therapy in Clear Cell Renal Cell Carcinoma, Cancers (Basel) 16.

Ref. 30 Bao, Y., Qiao, Y., Choi, J. E., Zhang, Y., Mannan, R., Cheng, C., He, T., Zheng, Y., Yu, J., Gondal, M., Cruz, G., Grove, S., Cao, X., Su, F., Wang, R., Chang, Y., Kryczek, I., Cieslik, M., Green, M. D., Zou, W., and Chinnaiyan, A. M. (2023) Targeting the lipid kinase PIKfyve upregulates surface expression of MHC class I to augment cancer immunotherapy, Proc Natl Acad Sci USA 120, e2314416120.

Ref. 31 Haggerty, T. J., Dunn, I. S., Rose, L. B., Newton, E. E., Pandolfi, F., and Kurnick, J. T. (2014) Heat shock protein-90 inhibitors enhance antigen expression on melanomas and increase T cell recognition of tumor cells, PLoS One 9, e114506.

Ref. 32 Caldas-Lopes, E., Cerchietti, L., Ahn, J. H., Clement, C. C., Robles, A. I., Rodina, A., Moulick, K., Taldone, T., Gozman, A., Guo, Y., Wu, N., de Stanchina, E., White, J., Gross, S. S., Ma, Y., Varticovski, L., Melnick, A., and Chiosis, G. (2009) Hsp90 inhibitor PU-H71, a multimodal inhibitor of malignancy, induces complete responses in triple-negative breast cancer models, Proc Natl Acad Sci USA 106, 8368-8373.

Ref. 33 Wang, L., Liang, Z., Guo, Y., Habimana, J. D., Ren, Y., Amissah, O. B., Mukama, O., Peng, S., Ding, X., Lv, L., Li, J., Chen, M., Liu, Z., Huang, R., Zhang, Y., Li, Y., Li, Z., and Sun, Y. (2024) STING agonist diABZI enhances the cytotoxicity of T cell towards cancer cells, Cell Death Dis 15, 265.

Ref. 34 Lu, D., Shang, G., Li, J., Lu, Y., Bai, X. C., and Zhang, X. (2022) Activation of STING by targeting a pocket in the transmembrane domain, Nature 604, 557-562.

Ref. 35 Pan, B. S., Perera, S. A., Piesvaux, J. A., Presland, J. P., Schroeder, G. K., Cumming, J. N., Trotter, B. W., Altman, M. D., Buevich, A. V., Cash, B., Cemerski, S., Chang, W., Chen, Y., Dandliker, P. J., Feng, G., Haidle, A., Henderson, T., Jewell, J., Kariv, I., Knemeyer, I., Kopinja, J., Lacey, B. M., Laskey, J., Lesburg, C. A., Liang, R., Long, B. J., Lu, M., Ma, Y., Minnihan, E. C., O'Donnell, G., Otte, R., Price, L., Rakhilina, L., Sauvagnat, B., Sharma, S., Tyagarajan, S., Woo, H., Wyss, D. F., Xu, S., Bennett, D. J., and Addona, G. H. (2020) An orally available non-nucleotide STING agonist with antitumor activity, Science 369.

Ref. 36 Davis, D. A., Shrestha, P., Aisabor, A. I., Stream, A., Galli, V., Pise-Masison, C. A., Tagawa, T., Ziegelbauer, J. M., Franchini, G., and Yarchoan, R. (2019) Pomalidomide increases immune surface marker expression and immune recognition of oncovirus-infected cells, Oncoimmunology 8, e1546544.

Ref. 37 Kowalewski, D. J., Walz, S., Backert, L., Schuster, H., Kohlbacher, O., Weisel, K., Rittig, S. M., Kanz, L., Salih, H. R., Rammensee, H. G., Stevanovic, S., and Stickel, J. S. (2016) Carfilzomib alters the HLA-presented peptidome of myeloma cells and impairs presentation of peptides with aromatic C-termini, Blood Cancer J 6, e411.

Ref. 38 Zhang, M., Wang, G., Ma, Z., Xiong, G., Wang, W., Huang, Z., Wan, Y., Xu, X., Hoyle, R. G., Yi, C., Hou, J., Liu, X., Chen, D., Li, J., and Wang, C. (2022) BET inhibition triggers antitumor immunity by enhancing MHC class I expression in head and neck squamous cell carcinoma, Mol Ther 30, 3394-3413.

Ref. 39 Stachura, P., Liu, W., Xu, H. C., Wlodarczyk, A., Stencel, O., Pandey, P., Vogt, M., Bhatia, S., Picard, D., Remke, M., Lang, K. S., Haussinger, D., Homey, B., Lang, P. A., Borkhardt, A., and Pandyra, A. A. (2023) Unleashing T cell anti-tumor immunity: new potential for 5-Nonloxytryptamine as an agent mediating MHC-I upregulation in tumors, Mol Cancer 22, 136.

Ref. 40 Dong, H., Wen, C., He, L., Zhang, J., Xiang, N., Liang, L., Hu, L., Li, W., Liu, J., Shi, M., Hu, Y., Chen, S., Liu, H., and Yang, X. (2024) Nilotinib boosts the efficacy of anti-PDL1 therapy in colorectal cancer by restoring the expression of MHC-I, J Transl Med 22, 769.

Ref. 41 Yu, Q., Dong, Y., Wang, X., Su, C., Zhang, R., Xu, W., Jiang, S., Dang, Y., and Jiang, W. (2024) Pharmacological induction of MHC-I expression in tumor cells revitalizes T cell antitumor immunity, JCI Insight 9.

Ref. 42 Song, T. Y., Long, M., Zhao, H. X., Zou, M. W., Fan, H. J., Liu, Y., Geng, C. L., Song, M. F., Liu, Y. F., Chen, J. Y., Yang, Y. L., Zhou, W. R., Huang, D. W., Peng, B., Peng, Z. G., and Cang, Y. (2021) Tumor evolution selectively inactivates the core microRNA machinery for immune evasion, Nat Commun 12, 7003.

Ref. 43 Shastri, N., and Gonzalez, F. (1993) Endogenous generation and presentation of the ovalbumin peptide/Kb complex to T cells, J Immunol 150, 2724-2736.

Ref. 44 Dickson, M. A., Okuno, S. H., Keohan, M. L., Maki, R. G., D'Adamo, D. R., Akhurst, T. J., Antonescu, C. R., and Schwartz, G. K. (2013) Phase II study of the HSP90-inhibitor BIIB021 in gastrointestinal stromal tumors, Ann Oncol 24, 252-257.

Ref. 45 Shi, J., Van de Water, R., Hong, K., Lamer, R. B., Weichert, K. W., Sandoval, C. M., Kasibhatla, S. R., Boehm, M. F., Chao, J., Lundgren, K., Timple, N., Lough, R., Ibanez, G., Boykin, C., Burrows, F. J., Kehry, M. R., Yun, T. J., Harning, E. K., Ambrose, C., Thompson, J., Bixler, S. A., Dunah, A., Snodgrass-Belt, P., Arndt, J., Enyedy, I. J., Li, P., Hong, V. S., Mckenzie, A., and Biamonte, M. A. (2012) EC144 is a potent inhibitor of the heat shock protein 90, J Med Chem 55, 7786-7795.

Ref. 46 Hong, D., Said, R., Falchook, G., Naing, A., Moulder, S., Tsimberidou, A. M., Galluppi, G., Dakappagari, N., Storgard, C., Kurzrock, R., and Rosen, L. S. (2013) Phase I study of BIIB028, a selective heat shock protein 90 inhibitor, in patients with refractory metastatic or locally advanced solid tumors, Clin Cancer Res 19, 4824-4831.

Ref. 47 Shin, S. C., El-Damasy, A. K., Lee, J. H., Seo, S. H., Kim, J. H., Seo, Y. H., Lee, Y., Yu, J. H., Bang, E. K., Kim, E. E., and Keum, G. (2020) Structural Basis for Design of New Purine-Based Inhibitors Targeting the Hydrophobic Binding Pocket of Hsp90, Int J Mol Sci 21.

Ref. 48 Tornoe, C. W., Christensen, C., and Meldal, M. (2002) Peptidotriazoles on solid phase: [1,2,3]-triazoles by regiospecific copper (i)-catalyzed 1,3-dipolar cycloadditions of terminal alkynes to azides, J Org Chem 67, 3057-3064.

Ref. 49 Rostovtsev, V. V., Green, L. G., Fokin, V. V., and Sharpless, K. B. (2002) A stepwise huisgen cycloaddition process: copper(I)-catalyzed regioselective “ligation” of azides and terminal alkynes, Angew Chem Int Ed Engl 41, 2596-2599.

Ref. 50 Neumann, S., Biewend, M., Rana, S., and Binder, W. H. (2020) The CuAAC: Principles, Homogeneous and Heterogeneous Catalysts, and Novel Developments and Applications, Macromol Rapid Commun 41, e1900359.

Ref. 51 Xin, Y., Liu, S., Liu, Y., Qian, Z., Liu, H., Zhang, B., Guo, T., Thompson, G. J., Stevens, R. C., Sharpless, K. B., Dong, J., and Shui, W. (2023) Affinity selection of double-click triazole libraries for rapid discovery of allosteric modulators for GLP-1 receptor, Proc Natl Acad Sci USA 120, e2220767120.

Ref. 52 Meng, G., Guo, T., Ma, T., Zhang, J., Shen, Y., Sharpless, K. B., and Dong, J. (2019) Modular click chemistry libraries for functional screens using a diazotizing reagent, Nature 574, 86-89.

Ref. 53 Kasibhatla, S. R., Hong, K., Biamonte, M. A., Busch, D. J., Karjian, P. L., Sensintaffar, J. L., Kamal, A., Lough, R. E., Brekken, J., Lundgren, K., Grecko, R., Timony, G. A., Ran, Y., Mansfield, R., Fritz, L. C., Ulm, E., Burrows, F. J., and Boehm, M. F. (2007) Rationally designed high-affinity 2-amino-6-halopurine heat shock protein 90 inhibitors that exhibit potent antitumor activity, J Med Chem 50, 2767-2778.

Ref. 54 Karttunen, J., and Shastri, N. (1991) Measurement of ligand-induced activation in single viable T cells using the lacZ reporter gene, Proc Natl Acad Sci USA 88, 3972-3976.

Ref. 55 Jung, S., Yoon, N. G., Yang, S., Kim, D., Lee, W. S., Hong, K. B., Lee, C., Kang, B. H., Lee, J. H., and Kang, S. (2020) Discovery of 2- ((4-resorcinolyl)-5-aryl-1,2,3-triazol-1-yl) acetates as potent Hsp90 inhibitors with selectivity over TRAP1, Bioorg Med Chem Lett 30, 126809.

Ref. 56 Tseng, H. J., Banerjee, S., Qian, B., Lai, M. J., Wu, T. Y., Hsu, T. I., Lin, T. E., Hsu, K. C., Chuang, K. H., Liou, J. P., and Shih, J. C. (2023) Design, synthesis, and biological activity of dual monoamine oxidase A and heat shock protein 90 inhibitors, N-Methylpropargylamine-conjugated 4-isopropylresorcinol for glioblastoma, Eur J Med Chem 256, 115459.

Ref. 57 Park, S. Y., Oh, Y. J., Lho, Y., Jeong, J. H., Liu, K. H., Song, J., Kim, S. H., Ha, E., and Seo, Y. H. (2018) Design, synthesis, and biological evaluation of a series of resorcinol-based N-benzyl benzamide derivatives as potent Hsp90 inhibitors, Eur J Med Chem 143, 390-401.

Ref. 58 Liu, Y. M., Tu, H. J., Wu, C. H., Lai, M. J., Yu, S. C., Chao, M. W., Wu, Y. W., Teng, C. M., Pan, S. L., and Liou, J. P. (2021) Ring-opening of five-membered heterocycles conjugated 4-isopropylresorcinol scaffold-based benzamides as HSP90 inhibitors suppressing tumor growth in vitro and in vivo, Eur J Med Chem 219, 113428.

Ref. 59 Muranaka, K., Ichikawa, S., and Matsuda, A. (2009) Rational design and synthesis of ansa-adenosines as potential antitumor agents, Nucleic Acids Symp Ser (Oxf), 5-6.

Ref. 60 Patterson, J., Palombella, V. J., Fritz, C., and Normant, E. (2008) IPI-504, a novel and soluble HSP-90 inhibitor, blocks the unfolded protein response in multiple myeloma cells, Cancer Chemother Pharmacol 61, 923-932.

Ref. 61 Combs, S. A., Deluca, S. L., Deluca, S. H., Lemmon, G. H., Nannemann, D. P., Nguyen, E. D., Willis, J. R., Sheehan, J. H., and Meiler, J. (2013) Small-molecule ligand docking into comparative models with Rosetta, Nat Protoc 8, 1277-1298.

Ref. 62 DeLuca, S., Khar, K., and Meiler, J. (2015) Fully Flexible Docking of Medium Sized Ligand Libraries with RosettaLigand, PLoS One 10, e0132508.

Ref. 63 Kothiwale, S., Mendenhall, J. L., and Meiler, J. (2015) BCL::Conf: small molecule conformational sampling using a knowledge based rotamer library, J Cheminform 7, 47.

Ref. 64 Lyskov, S., Chou, F. C., Conchuir, S. O., Der, B. S., Drew, K., Kuroda, D., Xu, J., Weitzner, B. D., Renfrew, P. D., Sripakdeevong, P., Borgo, B., Havranek, J. J., Kuhlman, B., Kortemme, T., Bonneau, R., Gray, J. J., and Das, R. (2013) Serverification of molecular modeling applications: the Rosetta Online Server that Includes Everyone (ROSIE), PLoS One 8, e63906.

Ref. 65 Nagapradeep, N., and Verma, S. (2011) Characterization of an unprecedented organomercury adduct via Hg(II)-mediated cyclization of N9-propargylguanine, Chem Commun (Camb) 47, 1755-1757.

Ref. 66 Lindsell, W. E.; Murray, C.; Preston, P. N.; Woodman, T. A. J. Synthesis of 1,3-diynes in the purine, pyrimidine, 1,3,5-triazine and acridine series. Tetrahedron 2000, 56 (9), 1233-1245.

Ref. 67 Sturm, T.; Sautter, B.; Worner, T. P.; Stevanovic, S.; Rammensee, H. G.; Planz, O.; Heck, A. J. R.; Aebersold, R. Mild Acid Elution and MHC Immunoaffinity Chromatography Reveal Similar Albeit Not Identical Profiles of the HLA Class I Immunopeptidome. J Proteome Res 2021, 20 (1), 289-304. DOI: 10.1021/acs.jproteome.0c00386

Ref. 68 Rammensee, H. G.; Falk, K.; Rotzschke, O. Peptides naturally presented

by MHC class molecules. Annu Rev Immunol 1993, 11, 213-244. DOI: 10.1146/annurev.iy.11.040193.001241

Ref. 69 Elias, J. E.; Gygi, S. P. Target-decoy search strategy for increased confidence in large-scale protein identifications by mass spectrometry. Nat Methods 2007, 4 (3), 207-214. DOI: 10.1038/nmeth1019

Ref. 70 Reynisson, B.; Alvarez, B.; Paul, S.; Peters, B.; Nielsen, M. NetMHCpan-4.1 and NetMHCIlpan-4.0: improved predictions of MHC antigen presentation by concurrent motif deconvolution and integration of MS MHC eluted ligand data. Nucleic Acids Res 2020, 48 (W1), W449-W454. DOI: 10.1093/nar/gkaa379

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A compound having a structure represented by a formula: 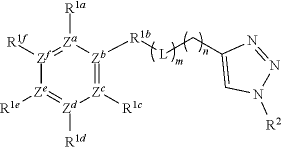
wherein each of R^{1a}, R^{1b}, R^{1c}, R^{1d}, R^{1e}, and R^{1f} are characterized by one of the following:
a) each of R^{1a}, R^{1c}, R^{1d}, R^{1e}, and R^{1f} is independently selected from hydrogen, halogen, hydroxy, an alkyl, and an amine; and R^{1b} is an alkyl or an aryl; or
b) each of R^{1a}, R^{1d}, R^{1e}, and R^{1f} is independently selected from hydrogen, halogen, hydroxyl, an alkyl, and an amine; and R^{1b} and R^{1c} are covalently bonded and, together with bridgehead atoms Z^{b} and Z^{c}, comprise a five-membered to eight-membered fused cycloalkyl or aryl;
wherein R² is an alkyl, an alkenyl, an alkynyl, or an aryl;
wherein each of Z^{a}, Z^{b}, Z^{c}, Z, Z^{e}, and Z^{f} is independently selected from N and C;
wherein L is an alkyl, an alkenyl, or an aryl;
wherein m is 0 or 1; and
wherein n is an integer ranging from 0 to 10;
or a pharmaceutically acceptable salt, solvate, or polymorph thereof.

2. The compound of claim 1, wherein each of R^{1a}, R^{1c}, R^{1d}, R^{1e}, and R^{1f} is independently selected from hydrogen, halogen, a C1-C6 alkyl, and —NR10R11, wherein each of R¹⁰ and R¹¹ is independently selected from hydrogen and a C1-C6 alkyl, and wherein and R^{1b} is an alkyl or an aryl.

3. The compound of claim 1, wherein each of R^{1a}, R^{1c}, R^{1d}, R^{1e}, and R^{1f} is independently selected from hydrogen, hydroxy, and a C1-C6 alkyl and wherein and R^{1b} is an alkyl or an aryl.

4. The compound of claim 1, wherein each of R^{1a}, R^{1d}, R^{1e}, and R^{1f} is independently selected from hydrogen, halogen, a C1-C6 alkyl, and —NR10R11, wherein each of R10 and R11 is independently selected from hydrogen and a C1-C6 alkyl and wherein R^{1b} and R^{1c} are covalently bonded and, together with bridgehead atoms Z^{b} and Z^{c}, comprise a five-membered to eight-membered fused cycloalkyl or aryl.

5. The compound of claim 1, wherein each of R^{1a}, R^{1d}, R^{1e}, and R^{1f} is independently selected from hydrogen, hydroxy, and a C1-C6 alkyl and wherein R^{1b} and R^{1c} are covalently bonded and, together with bridgehead atoms Z^{b} and Z^{e}, comprise a five-membered to eight-membered fused cycloalkyl or aryl.

6. The compound of claim 1, wherein R² is a C1-C10 acyclic alkyl, a C3-C12 cycloalkyl, a C1-C10 acyclic alkenyl, C3-C12 cycloalkenyl, or a C5-C8 aryl.

7. The compound of claim 1, wherein at least two of Z^{a}, Z^{b}, Z^{c}, Z^{d}, Z^{e}, and Z^{f} are N.

8. The compound of claim 1, wherein L is a C1-C6 acyclic alkyl, a C1-C6 alkenyl, or a C5-C8 aryl.

9. The compound of claim 1, wherein n is an integer ranging from 1 to 6.

10. The compound of claim 1, wherein n is 0.

11. The compound of claim 1, wherein the compound has a structure represented by a formula: 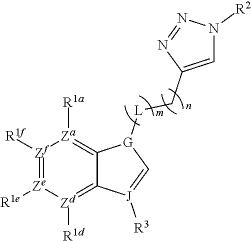
wherein R³ is hydrogen, halogen, an alkyl, or an aryl;
wherein G is C or N; and
wherein J is C or N.

12. The compound of claim 1, wherein R³ is hydrogen, halogen, a C1-C10 acyclic alkyl, or a C5-C8 aryl.

13. A pharmaceutical composition comprising a therapeutically effective amount of a compound of claim 1 and a pharmaceutically acceptable carrier.

14. A method for the treatment of a disorder associated with uncontrolled cellular proliferation in a mammal comprising the step of administering to the mammal a therapeutically effective amount of at least one compound of claim 1.

15. A method for the upregulation of MHC-I expression in a mammal comprising the step of administering to the mammal a therapeutically effective amount of at least one compound of claim 1.

16. A method for upregulation of MHC-I expression in at least one cell, comprising the step of contacting the at least one cell with an effective amount of at least one compound of claim 1.

17. The method of claim 16, wherein the cell is mammalian and wherein the cell has been isolated from a mammal prior to the step of contacting the at least one cell.

18. The method of claim 16, wherein the cell is mammalian and wherein contacting is via administration to a mammal.

19. A kit comprising at least one compound claim 1 and one or more of:
a) at least one agent known to increase MHC-I expression;
b) at least one agent known to treat a disorder associated with MHC-I expression;
c) at least one agent known to treat a disease of uncontrolled cellular proliferation; or
d) instructions for treating a disorder associated with MHC-I dysfunction.

20. The kit of claim 19, further comprising a plurality of dosage forms, the plurality of dosage forms comprising one or more doses, wherein each of the one or more doses comprises a therapeutically effective amount of the at least one compound and the at least one agent.
